SEQUENCE LISTING (9 sequences)

SEQ ID NO: 1 - DNA, 24023 bp, Toxoplasma gondii
atgcctcacagtggagggcggagagctgttgctcccatttaccctctcgatctggcaggtcagtccgccgtttcgttcagacttccttctcgtctgggttctttgtttcacacgcctggccgcgtctcctctccgcttcttggtgtgcagcctcggtttctcccctttgatcagctgtctcggttgcgcttccgccccgtgcgtcgccttccgtgtttgcttccaattttctcctggcttgctcgtgtgttcgcgcccctccggggagaccggcctgtctgctgccgacagagaggcagctgtgaagggcgtgatcatcaactgtctccgacggagagacacgacgtctgtgatgcaagcaaatgagcgcgtgtatgcacaggtacccgcgcatactgaaatattttcgtatctgcagatccacaggcgcatgcatgcgcccaactgtacccacgcgtgtttctacatagttgtggagaggcacatgcgtctgcatgtgtgcacgttgtctattcttgcgaacgataaacctggtggcgatccgtgtgttattttcaagaagtgatttcgacgcaggccatctcgctcgtcgctcgtttcctgtgtttgtcggctgctcgcaggacgtctgagacctgcaatgctggtgttagccgatgggactgagtttctcggatactccttcggctacccaggcagtgtgggaggcgaggtcgttttcaacactggtacgtttttctcgaatttgtccagaaacgctgacgtttggcgtctctcctctccagaaagggtgcattcggtctatccgctgtgtgctgcagcgattggtcctcctcttacaagcgcgtacaccaccctatgcagcctcatgcaccgccatctgtcaacgcgtgtggggaccgccaccacacgcccatgtatctaccttcgataaacatatttatgcatatatatatatatatatagcatatatatatatatatatatagcatatatatatatatatatatgcatgtagatactcaaaatgcatgcatattggtacgtctgttcacctgtatttttctgcgtgataattagatacccctgggttgcgtcaccatgagctgtgtatcgttctgggtgcatgcgttgttgcgggagtgttcgtgcgtcgggaaaggtagtggccgacttcttgtctttggcgtggttaggtatggtcggctaccccgagtctctgacggatccttcgtacgaggggcagatcctcgttctcacataccctctcatcggcaactatggcgttccctcttcggaaaaagtgagagcagacagcaagaaaacgaagagactagcagaaccccgtacttcgtggctgatccacatcagtgagagtcgaggagggagagtcggatttctcttgcgacgcaacgtcacaggaaagcaagtctggcaggggtctccgctttctcacatgccgaatgcgcacacaaatcattcatacgtcactacaaaactgagtcctacgtgagaagagcaacgtcacctctccgtgagacatatacgtgtttctatatacatatatacatattatgtatatatttatattcaaatatatatatatatatatatatcaatgtgcatatctaagtttatatatgaacgttcatctgtctttcgggagagtatttccctaaatggcagatgctaagacgcctgtacacctgcgtgcaaaggtgtttacgcgggtgtctacacgtgagcgaagtgattgctacacacatgtatatatatatatatatatgtatgtcgctttacagtgtttgtttttctgtacatctaaagtcgtctaggcatcgatatgcgatgtgcatttcagcgtatttcgtgtgtttcatttccactcttcaggatgagcatggcctgccgaaatactttgagggcgaccgcatttacgttcgcgctctcgttgtggcggactacgacaacgcagccgtgacggcacactttcgtgcagagaacagcctcagtgcttggatgaacactcacaaagtcccggcgattgcaggtgcgaaaacatcgaggccgaagtgtctgtagtgtcagatgcgcctctggacacgacatcctctttcgatgcttgtctttgattttcacttaattttctcttcgcaagtctgcgaaggaacgcctgtctgtacatctcgatgcccaggtggctttctcggccatttggaggaactgtcttggatccgcgtagtaggcatgtatgagggagcagcttctctttcttctgaattgtgttccacttgtaagtcggttcgaccacgagcagtcaagaggctcttaccgtgccaccgagtcaacgtcgcccatcacagaccggcttttgaatgcctttttcttcaggctccattaatgcacgtctcactgaatccgtgctcgtcaatttggacagctagatctgtgtagtccctagagactaacttttggagggagactaacaatacagctaggttgctctacctcggctatgtttaacgcatagttcacggatcactgttgccactggtccttacagagagagcacacgactgctcacgtgcttgtcatggagacacatctcgatcgtgtatgtttcacttcagttctgcagagccgttcagtgtatgtctgccatgacggggaggggtatctcaagggtgtattttgaactgtatttccacggtgtacggggcttgaagtactggcgcatctatgtctggaggacgggacgtggtttagttcgtgtctcagtgctcaacagcgtccggatctctggcatgctgttgccatcctgttagttgctggccttcgcggctactctttctcctttaggtgcacctccagtgtctccagttcagctggtgttcccttgccgactgttgctgctctgtcagggctttgtaggtccccgcctcactctcgtttccgcttctctgttttctgcgtccgtgtgtgtctcacctcttcctcttcgtttccatctcttttcttgtctcgcctttccacggctctcgtttgctcttgaaacgctgtcggttgtcgtcgttccgcctcgttctctattcggctctatcttcgtcgttcgtcttttgcttcttttcgacttcccgactcctgcctgtttccggctgtatcgttttctttttcaaggagtcgacacgcgagcgttgaccaagcacctgcgcgaggtcggctgcatgctgggcaagatcgtcgtcctgagcgaagaagaagagcgtcgatccggcttgtcgctctcggctctcgccgcgcttccctcagcgactgcagcagagcaacgaggagagaacgacgcgacggtgacgcccgacaaagcagaggcccgcctaagagtggagaggcgacaagcagcgctcacgatgtgggaggaggcgatccgcaacaaggcgaagaacctgccatgggaagaccccaacaaagacaacctcgtcgccctcgtttcgcgaaaagaagtgcgcgtgtacaaatctactgtcgtggatccggtgagtgacagagagcccagggaaagacgtttcacgtgcgaaagcgaaacgcagtttccacagtccttgtttagatttagcgtgggacatgcacaagttgggattctgcggaatgccgctatctggggaggggagtatagacgccgagttcaactcctggcttcaatctcccctcaatcgaggcgtagcgcaacgcgctcgctttgcactggtttcgccgccccggaggtccgtgctgagctgtacgtacatccggggtgtgtatgcccgttgcgatcgcgtttgagtgttttcggggtctgactgtgcgtgtccggggcctcccgggcggcgcctgtcgaatgtgcggctcggctttgcccgccttctttctccaaagtgtgtctggactgcctttctgtgttctgtcggaatctcaagtctgggctgtcggtttcgttgcctgaatctgcagaatctccgcgacgtcctcatcctctgcgtggactgcgggatgaaatacaacatctaccgccagcttctccatagcaaattcgagcactgcaacatcattctcaaggtacagctgtcgctgctctgactgcattgactttgaaactctcattcctgatctgttagctctcccggcagttcgcttgtattttctgtttcctgtggcgcccacccgtcaattcccctttctgcggcgccgagcgctcttcagctggtactgggaaatggagatgtgtgtccagcagtatcaagacatacagaagtatactgatgtgtacacgtgaatctccattctgtttatgcatgcatgtctctctctctctctctctatatatatatatatatttgtacatgtatttatgtgtgtgcgtgcgtttgaatataagtacgtattatctacaagtttgcatgtttccgtacgaatgtccgcaggcgtgtgtgtctggttacatgccgctgaatctcactattcaattaaacagttgagtgtggagaggtaagcgagactgtgactgcgcaggtgtgaaaccgttcaggagcgcatgcgctgtgcgtgtctctcaggtggtgccgtgggatttcgactttggcaacgacgaatttgacgggctcttcatcagcaacggtccaggcgaccctgagagatgcgaaaaaacagttgctaacattcggtgtgtgctgcagagaaaagccgattcgttcgccagagtcaggaacagcacagtggcgcatttccatttccgtcttcgctgtagacacgcaaaaccatcgcgatttgcagattggttgagcttgctctctgagtcgcgggaaactgttccttccgttccatggcgacccaggcacagagaagcgtgcatgcaaaaaacaacgtggagtctctccgttttgtctctgctaacttagtataactttagacccggcaaacagcgacatgcacgagttaaacgcgtagtccatctcttacatgaaatggactctttagaaagcgcaagacggtgcacgctacaccagcctcgttcgtaggcttgcgtattgagttcacgcgattgaacaccgaattgtcgaggcgggaagttcgcgtctaccacatattcatctgagttccatctcgggttgctccggttcgggccaaggtgactggaatgcccggtggctgccgggtactatgtgcttccccgcggatctcccatgttctcttgtgtgggcagactgcgcagcactgctggtggctgcgtaggttcatgcccttacaatgtaaatcccttttctaccgttcgttcttcctcccgttttcagacgcgtcatggagcgaaagatccccatcttcggcatctgcctaggaaaccaacttcttgccctggctgccggcgcgagaacgtacaaaatgaaatacggaaacagaggtaagcgttgtcgttcgtcggtcacactgattgtacgccgtttcaggtgtacgtacacctctttcaccggcagcgaggcgccggtccagggagcgtcccgcgacgtgggtggcaggccagtgactagcgacggcgaggcgaagagggaaaatagcatctccggactctcatttctgttttgccgttgcaggaatgaatcagccggtgatcgacttgcgaacgtcgagatgctacatcacaccccagaaccatggctttgccgtcgacgagagtaagcggcgacaacattccatctgcgaaatctaggtgccatgacctccatatgccgttatcgtagacataatgttgatatgtagaatgcatatggatacgtcgagagaggtcagattcacttttgtagatgtagacggctataaatcaaagtggttcatgcatctgctgttggttgtattttgacagcattgtgaaagggagtggtgttcgatagaacgcaaatgtgacgcaattgtatccgtaagaccctgagtttcattataagtaggtattgcctctgtacaaatgatccgcgagccagaactgtataactcaaaccgaataaacagagtgtctgtcgctgtgaatacgaatggtaacagtaatccatatattgcgcctagaacgtaaccgatgtggaataacgtaaaaatgcgtgtttgagccctccacgtcagcggtctgtcgattgtgaattagcgaagccgacccgtcgatgaagttcaacagtcagccagatattcagtctctagaacctccacaaaggatgatgtcctccaggtagcgaagataccaactgtggtgttggagaatggcgtttcaaagccggtagcgttgcagacactcttgtgtcctcgtgggagtccgtgttcgaggcgggcagttgctacagcaagtagatgcgtagaacgaagcgagacctccacgcggggatttaccttttgacatcagttctggaagactgcagatcctttgcacagatgcaatatctgcttacggtgtggcgcttacaatacatggtcgagctatgctcctgtcggtctcagtgcggccttcgtcagatgacagatgtggacacctgcctatatgaccctgtgttgaacctctcgccgtttctgcaggcacgctgcctcgagatttcctgccgctctttgtaaatgcaaacgaccgttcgaacgaaggcatcatccatcgcacgctgccttttttctctgcacagttccacccagaggcgtcaggtaaggcgggcgatacttcttcgactgaaataccgacgttgcagatcagcgacatctctttccgttggcgtcgtttcaatgatcagtgcttgaaggcatcttggcagcgtttcgtcggaccaatacggcggaactggagctcgacggtacaaggaatctgtgactgccgatgctcttctcgatgatgcaggggcgacagttcctttcatgagggaaacatgtgcttgtgtcatcagttcagttcctcataactggaggcatctgttgggtcttaaataaagccgcccgctaagaaggatgctgccttgcgacgcaactcgtgtgtcaccaactgcatcgcgatgggagagtttttcctctgagacaaaacgaggatggacctccgaagttcgtgtacgcagtctgcgagtcagtggggtgccgacgcacgagacgaagacgagccactcagagattattgtttctcacttttctgcctcggcgaagaagcagcgcatacccatcggtgcccctccgctgggcgtctcgtgcgttccctgtcgctggcgcggcttcgtcaacacagtgcaccgattttttcttctgctcgtttgtgacaacctagagagctccagtgagatcgaggagtggcgcgagatgtgagtttcacgcggtggaggccagatgatgtttcgtcaagagctgcgccgcagtctaccacgcagcgcagaccccgtggggtgtgtccttcgcgcgattctttcctggagtctgtggggtgtttatacactcggcgtccctctgtctgtgccttctggctcaggtggtccgacagacacgttttacttatttggcgacttcatcgcctcgattatgaaggcgcagacgctgaagcaggtccacacgactccgttctcctttccgcagaagttccagaaagttctgcttctcggtaaggccggtgttcctcccgcatcttgagggaaggcgagttcttttaaagtgcagaaagccctttgcgggggtcatcagagaaggaacgccatcggctgcatccttcttggtttctcgcaaatgcgtctgtggcttggccgtcgcgtggttccttctgaacgcccgtgtggaggtttcccgacgctgtcatctctgccaaggtgtcgtcgtggacgttcaagagtgcgagagcggccctgccgactgtcgcagagagcgcggctcatgtcatgtctcccttgtgcattctccctgagcttttcgcgggctctctttcgacctctgtctccgccgggatcatctgtttaggaggaccgtgtcgtggtgactgtgccggaggctctgctcgctcaagtgaggaggtcggggtaaggcgggaagtcggctgtgtgtgcgtggtttatttgcttcgtagggagcggaggcctgagcatcggccaagccggggagttcgactacagcggctctcaggcgataaaggcgctgaaagagcagaacatctttgtcgtcgtggtgaaccctaacatcgccacggtgcagaccagccagcacatggccgaccgggtgcgttgaccgcgcagacggattgcgcagagtggcgtcgagggagcgcgaacgaaaaaggaggacgcagacgccagagaagacacagagaatcagagagacagcgagagacagcgagagacagacagcgagacagaagatcaatcgggaggcaaggaggagagcgaggtagagagaaaccgcgagaaacacagagaggagcagagagctaacaagacagaaacaaagcgtggtgcaggaagacgcagatgagagggagagacggagcccagagcagagacagaaagacagagcaaagagagacacagacagaacagagagagagagataagggtggaggtgaaggaataaaggcggggcttgagcgaacggtgaagcattaggccgtggaaggtgacgaaagtgcgatggaggcaggatggtctttgtgaggccttctttgtcggaagaagagagtggacaccacgtatttccactgctgcgttgcgtaacgcgtttatgagagagtactgcggttccccagagactggcggaaaccaaggagtcggcaggctttctcgggtctctctcccctctgcgttgtgagtttcctaacgtctgtcgacttcgggaagtccgaaactctccggagaagacacacgcaactccaaactgcgagtggagtcgagccgatccagtcgagtagtagcttgcatgaaatgtgtcgagccgggtgcatgcaattcattttttcaggtgtacttcctgcccgtcacggatgagttcgtgacgaaagtcatcgaaaaggaaatgcccgacggcattctctgcacattcggaggccaggtgcgtttgcgtgtgtgcaacggctttttcctcgatgaacgtaactaaatgcgcgaagatataacgaggcctaggcactcgcaaatggacaatccatccatatgcatatatattcatatatatgcatgtttaatatatatatatatatatatatatatatataataatatgcgtatggggctggccattgaggagatatgtatgcagctgttcagaggagtagcgtaggcgaacacgtttgcatgcatagagttctggacgaagcacgtgatcgtgcggatatatgtaaatgaggttgtgggtgtatgtggggacagcgatatctggacagggtacggctttttttttcactttatggcatgttcagacggccctcaactgcgctgtgaaactccacgaacaaggcgtcctggcaaaattcggctgcaaaatcctcggcagtccaatcgaagtaagaatgtattcatatatatacatccatatatatatatatatatatatatatattcgtaactgcataaatgcacatattcatagtgatacatatatatatgcacatagacatatgcaagtacatccgcgcacacatatgtccatatatgtgtgtatatccatatataaatatatatccatatatatatatatatatatatatgtagagaaatgtgtgtgtgtatcagtgcacgttacgatgcacaaatttgtatactggtgtgtatttaggtatccgcgcatttcgagtttcgtgggaagcacgcggtgtcttgtttgtcttttttaggcgatcattgcgactgaggatcgaaaggtgtttgcggcgaagctggaagaaatcggagaaaaagtggcggagagcgcggccgcgacaaacacggaagaagctgtgcaagcggcgaaggccattggctaccccgtcctcattcgcgccgccttcgcactcggtcagccaaagaagagaccggggagtgaagacgagcgaagaaaaggggcgcaaggtcgacgagagcaatgagaggagacccagaaaaggcgcacacagaacggatgcagacggaaacagacgaggcgacaaaagaggcagtcgatgacagagaagaatgcgagggagacggaagcaccgaagacgaagggaagagaactggccagaaacgaaggatacggatttcacgttcaagtgatgtcaacttgcgtgtggactatcgcaggtgggctcggatctgggttcgccgaggacgaggagaccgtccgacgcatttgcaaggaagccttctcccattcttctcaggtccggtcaacttctcgtagtgcatgcgtatacgcacatcagagtaaaatttgcatatatatatatatatatatataagtatatgtatgcacacatatatgtttcagttcttatctgcgggtgtacttgtgcatgtccgcaccggcgtcgtgtgggcagatgtgaacgtctggagagagaggtcgcccgttctgaatgcgcgtgcatgcgtttctgacaggcgtgtctgcatggctgcggaagccgaatgaaaccccacacttccgaaggaatttcgcgtttcagccggttcgtttgcaggccgagtgtcgtcggggaggcttcctcgttgtgttggtgtgtgccggtccagtggttctcgcgcgaagccccgagtgcgtcccctataagactgaaaagcgccaagtcgagctggcggaactctgagaatttacgttttttcaggttttcgtggacaaaagcctgaagggctggaaggaggtggagtacgaagtcgttcgcgactgcaagaacaactgcatcaccgtctgcaacatggagaacttggatcccctcggtgcgtctcgtctgtgtgcatttgccgtgtttgctcttcaactgtggcttctcagctctgtcgaggggtgcgctgcggaggctcgctgtacaccacgaattgtttctggcctctctgttaacgagcgttctgaaacgaatccccacacaagtgcatgctccctctcgtcctctcgctgcttgactctgtcgctgcgggggaatccctcgtaccgtgctctcgctgtgacgacctctgagtggacgcgtctctctcggacttctctttgtctctcgccagacgcagtgattccaagtctctctctttttgtgcagcgctgtctctgacgaacgctgtctatcggcgtatctctccttctccgcttctccagagcttggcacttctctgtataaagatttacatatatatatatatatatatatatatatatatatatatatatatatatatttgaatgtttttgtatatgcactccatctggataattcagtcgtcgaactctattgtcacagcgttttggtcacttaaaccggcgtccttcgtcgatggtttgcgcatgcgctttttgcgtccatgtctgtacacccgcgtgtggattttttcccctcaggcatccacacgggagattcgattgtcgttgctccttcgcaggtgagctgtcttcgcttgtgtttttctgtttttcatctctctttatacatttctcggtccgatatttctgctctctcatggcctctcttttgccattttcctcgttgttttctcttatccctcgttcttcctcttgttatgtcttcttcttatcctttgttatcttttgtccgcgttggtctattgttgtctccgcttcaaatcccttcgtctcccacacaagcgcgcgttttcttctttgctctctctcttcagacgctgtctaacgaggactactaccgcctgagagataccgcgctgaaggtgattcgtcacttcggcatcgtcggcgaatgcaacatccaatacgcgctcgaccctaactcggagaaatactacatcgtcgaagtaggtgacaagagagtgcaaacgaaaaaccacgggaggcgaagggaacgcggtcttacagaggatgatgccgcgaggaaaggtggacgaacaccctgaaacaggagagaggaaagcgagaaccggacaggctcacaccaaagtcagacagaggccacagagcgagaaggacagggaggtgcagaagcggagagaccggccagaaaagagagagacaggggcggagataagtgctacagaaagtcgagagaagctttctcgatcctagctcaggttcgatgtcacctgcctcgcgctgtgcggcatggccacacgcatgctgaaagtcaacgagaaggtcgccaagcagttctacaaatcaagcactgtccagacagctgctgacgggcattccgtttttccttttgtgggcgtttgtgtcgctgtctccgacgttcggacgtcccgcgactacagcgtctctgactttccagacgcctcgctttctcatctcttctcccgcttcgctgtcttgtgggccttttcctcaccgcaggtcaacgcgcgtctctctcggagcagcgccctcgcgtccaaagccactggctaccctttggcctacattgcggcgaagctcgctctcggtaacgtttgtctcccttctgtttcgtcgaaacttctgagtggcttttccgaactgttcgtcccagagtcgatccgcatggcccgctcaaagcccagcatcccctttcattgctgctctccttgatgcttcaagcgtgtctgcacggccttccttctgttcttaaattcgcggcggtgatgtgcgtttccgtctaatcttctttgtctcgtcggctgagtgaggtggtgcttcggtcggttttcatcctcgagaactggcagtgtgtgtcgcatgttttctctctccatgtaggctcgactttggtggagctcagtaactccgtgacaaaagagacaaccgcctgcttcgagccttcgctggattacgtcgtcaccaaggtgccgaggtgcgaggacacaacgcacacagatagaagttccatagggactcccaggacaccagtggtttcttccctcttctctttgtggcccgactgctccctcgttctctctttcctgcctctcttcatctcttcctctgtttctctcttcgttgttcctgtccttcgccgtctcttccgctgttcactgttggggtccgttccagttcggccgtcgcttgcgtgtcaacgtgtgtatggttctctctcctcaacgtcaccacgtcgccagcgtccttgcccaaaattgtttctgccttctgcaggagctcaacgccttctgttagatgcctctctgacgcatgctttgcatctctgcactcgatagagacttgccgttttgaggaagaagtttccagagcgtctgagagagtgtcctcgcgcgtctctcgacgtccagcgaggctcgcgcctcctggcggtgaacagattcgtgttgaatatgtctttctgctctcaggtgggatctgcgaaaattcgagtcgtgcgaccccctgatgggcagcgcgatgaagagtgtcggcgaggtcatggccattggacgcaccttcgaggagtctctgcaaaaggcgctcaggtaaaccacaaagttccaatcatcggcatccgtcgacaaacagttgtcccagttgaagttattgtagccacatctctcttctccgcatttcctctctcttcctcttctccctcttgttctccctcttggtctcccttgtgttctctttcctgttctctgtttgcgacgtttcttcgactgtgtctcgctgacgtccgttctggtggcctgcgttttcgcctctgttcttcagaatggtggacgagaaggccggcggcttcgacgagtcggtctgtcactttttttccacggacgaggactgcgcgccttcgctgcccgggtcagacttcaagacgtcctcctctggagaatgcatgcgtggcggctgcggacgcacagactccggcgccgagcggcaggctgcgctgctggaggcggaacttcgccgtccgtcaccgaatcgaatctgggcgctggcgctcgccttccagctcgggtggacggtcgacgcgctccacgagaaaacgaaaatcgacaaatggtttttaagcaaacttcaaaacatcaacgacatcaagcgacagctcacccagctcaccctcgatgacctcacgcgcgcagatttcttctacatcaagaaatacggtaaacgccttcgcgcgctgtcgagacactggggcatgtggctcgggtgtcttcggggtggccgtcaaacagtggggtgtttcccagtcgcatttctcactcgtttgtacatctccgaaaacacggcgacgatgcgcaagccgaaggggacaagagacaagcgatgctccatgttttcaaagctcctgtctgttccccgtctcttgtggcaatcgtcgaagatacgctaaccgcagactggtggtcaaatgttttttttcgttttgggatccactgtggttctcatcttctcgtcgtctcttgtcttcgtctctcgctctcggcttctcgtcttctactcggtctcccctgctccgtttcttctcctcgtccgtgtctcatgttcctcttttcttctcgccttcttcgcttcgtctgtgcaacgaccagactgcgacaatccctccccgttagtaggataggccgcaggtttctctctgtttcccaggattcagcgaccgtcagattgcgcagtacttgatgaattcaccgagcgcggctgcgctgtcgcagttcgacgtgcgtcgtcggcgactgcacctgggcgtcagaccgtcggtgaagcaaatcgacactctcgcggccgagtttcctgctcacacgaattacctttacttgacctaccaaggaatcgacgacgacgtctcgcctctcgccgccacgccgtccgtctcggcggtcttcgctggcgcgcgagccgagaagagagaagaagaaaacgcagagacatgcagagacgacgaggacgaaagtctcctccgccgcctgagcaaaagctccagcgcgcggcttagaaccggcgaaggcgacgcacccggaaaacaatgttttgtggttctaggtaggcgacgaaggcaagaaactttgaggatggagagacgaagaagcattgaagggagagaaatgcaaagacgggagagagatcgagaaccggaggagaagagagaggaatggcgacagagatcaagggacgagttggagacgatgcatgaagcaaaggcgagcaaaaaaggcgattcacggaagaccagaatcagacgacaagaacatgtctttgtttatcgacgggcacacctctttttcttttctctcaaggacctgtcggtgcttgcgcaccaggtactgcgatatatatatatatatatatatatatacatgaacatccacagatatgcttgggtagatacagaagtacatatatatacatatatatacgactattcatgcatatgcatgtgtatatgcttttttgcacctttttgtgcgttggtattttttggtgtgtttcggcaggctgcggctgctaccgcataggatccagcgtcgagttcgattggtccgctgtttcgtgtgtccgtacacttcgctctcttggccaccacgcgattgtggtgagtattcgccgcctctcagtggtagatcacttcgcaaacgtttcggtccttaggtcaaggaatttgcgacaagcctctgcctcaggttcctcgtgaatagcctcagactatttttaaacaaatgcagcgatgcattaacgcatggggacactgcatgtgagatgagccacggttccgcgaatatctttatggatatgtatcaatggatacataaatatatatatatatatatacatatattatgtacgcagtggaggccatgtagaaatgggcatttttacctctattcacgaataagagtaggtctgtgcatgacgacgacggacatgaaagtgatgctagacagtatatgagcatttaaaaggtaattattcataaaagtgtttattgaacgttgttcgtcctgctcttgtcaattcgcattccgtggaacaggtaaactgcaatcccgagactgtgtcgacggactacgacgtgagcgatcggctgtatttcgaagacttgagcttggaaacagtcttgaacatctggtaggtttctcacgcgtagctgtccgattgtgggctctccgaatataagccgaatcgcggtgaccagcctggatgtcggccttggacgtgtgtctggcccctcagattccccgaacattcagagagagctcacatggcccgtaggtttggtgtctctcagtgtgtcatgcagtgcatcaaagttcgttctttgaattcgcacatcccgtcgatgtctgtgggtccgccttggatggcgccgcctcgttggtatttgaagtcccaaataagaggcacagaaatcactgttttcgctgtgtggaagacacgttcttttgtgtgtcccagatttctctcttcttgtctttcttctcacgcttctcttctgtcgtcatctgtgcctcgtgcacgcgtctctgaactcgtcgtattctcctctcttgctcgtgtctctttccctcctgtttgactttcttttccgtctcgcttcgcttcccttgttctcccgtcttgccctcctgcttcacgcgttgtctgtcacgtctggctttcgttggtcgcctctgttttccggagttttctcctcctccactctcttctttatctcagaaagactttgaagatttaaacaggggtccacctcctattctacactccggagaagaagtcataccttcccgcgcggtggtggattccattcgctatcctgtgtctcctgcctactagtcaagagatgttgcgcgagtgtgtgcttctcgttcgagggtcctctgtcgtgcgtttttcgttcgtttccagggacattgaagccccggcgggagtgattatttccgtgggcggccagacaccaaacacgctgtgctctgcactggagaagcagggcgtgaggatcgtcgggacgagtgtggcggcgatcgactgctgcgaggacagacacaagttctcccggctctgcgacgagctgaacatcgaccagccgaggtggaaggagttcaccgaccttcgcacagcgaaggccttctgccaagaggtaagcggaaacacatggttcattcggggcgaaacaagagaaaacgggagatggagatggggacgaaaacggagacgaacgagaagcaatcaaatggaaggcaaagcagccgagaaatagagagacacggagacgcagaaatgtgcgcagaggacagcgaaacgcagatgaagcggggggagacagagcggtcttcaacagcgcgcagtcgagttgaagaagagacgaacgaagaacggacgtcaaaacacacaaggggagcgaaccttggaggactgataacagaagacgaaaagtcgaattggaagataggaagtcgcaggagagatgacacaacctgtcgaggttccaaaggggtagcgtgtgcccaagagcaaaagcattcgccggatgtccttacaacgtgcttgctggcgcgacgacaccatcaggttacctcgaaagtaaataccgtttctgcatttttatcgaagaggtcttctgtctctgcttctggcctacgtaaaataaaaggcttgctctgtgaaagcccctgccggtccacatgttcgtaccgcacacgcttacagtggtgtcttgcatgcatttttcgctggccgtttttcttcctttcccttcgcgacggtctgtacgtacgcccgagaccccgttggtccacaggcgtccggcgcggtggtgtctctggtactccggcctcgtcgaacgtatgttttctcgcagtttgtctggcacgtgtttgaaaggctgtggattcgcaagtcgcgttttctgcgttcgttctcgctcttctctgcaggtcggctacccagtcctcgtgcgtccttcctacgttttgagtggtgctgccatgcgagtggtgaccgacgacgagcagctcgacgccttcctcaaaatcgcagctgtcgtcagtggcgaatctcccgtggtcatctccaagttcgtcgagaacgccaaggaagtaagaaaacgacctagacaggctcacgacttccacgcttccacgcatatccctgtatcgtgttcgtatctactttcctgtgggctacatgtctccgtgttcctatgcagtgatgtcagagatcttcctcgaaatactctcttcagctgcgtataggtgtatacttgcatgatatccatttatcttttgcttgcgtatctatgtagatggaaatgcatccgaatgggatatatatatatatatatatatatatgcgtgtgttttcttataattatgtatatatatatataatatcgatgtgtgcaaacatgtgatctccgcttagatggggaactcttgagcaatagtgaggcaaacgaaacgaagttgtcaaaatcagcaggaaccaccgggatgaacctggtgcatgcttgcgtaagcataggcgtgcaagcatagtttcacatggtaggcaggagccggcattgttcggatgtatcgtcctctcctcgttccttccgtggaaactgcctttttggcttgcaggtggaatttgacagcgtcgcttgccggggggaaatcgtaaactttgcaataagcgagcatgtggagaacgcgggtactcactctggagacgcgactttgattctccctggacagaagctttacgtggaaacgattcggtgaggacgcatgaagagtgtgttgtgacacccgtccccagtgcgcgacagagaaaaagagacgaactttccaggaacgcagagagtcttcaaagattccgttcatgagtcgcggctggtgcatctcgcgcccattgttttcaaggacgctacgcgcccgaacgagtcgtgttctgtctcagcgtctccagttgctgaatgccgtggtgtcatcccagcaaacgcaactgtcgctcttgtcgagagcgcgagaaagagaacatgcgaaaagagcgtttgaagggagtggaggcggcacctgcatgtaatcgagagaaagaataatggcttagttgatgaaggacggtggagagaaagtgatcgaacttgaggcgcagtgagggcgcagagacagcgacaaagacatgtgaggatatccaggcaagtgtgatgggagagtcaggtagatatttgccagtggacaatattgctagaaatggagacaagatggatgcagggtgaacggacagttatcggtcggggtatacgagaaataaatgcatgttgaatgacttggatagatgagagagagagagggacgtagtggttccttggccttgcgccgccttgtcgtgaagggtcgacaccagtcagatggagattctgtcgcccacatcttctttagaagaattgaaaaagctgttcttgtaagttgtgaaggcacaaatgttttttgcgtgcagtcgcgtgaagaagatctcgcagaaactcgcgcgcgcactccaagtctcaggttcgtttttctcatacactatctttcgttgattgctcttcccctgcttctcctcaaaaacctttctttgcgtcgtgcgttcgatccggtgtcttctgcctttctgtcaggtgccaactgtacacggttcgagtttctgtcttctgagtcgagtgttcttcttcaccattttttcgtcggacttcgtgtcgctttgtgttatccgacagtcgatagacctcttttctccatcacgagaaagcgacgacgttgcctttcctatcgcagctcacccagtggaggcaaaccgcattggcggatctgcaattccaaaccagagttcagaggcgcctgagactgccggacgttccctcgagttcgtgtcagctgcatgcgttgcatgcgcatgcacagcgccagggacggggcacctgcgggtccgcttgcgagagggcgtgctgtgcttttctgcctttcttcaggtccgttcaacatccagttcatctgcaaacagaacgacgtgaaagtcattgagtgcaacttgcgagcgtcgcgtactttccccttcatcagcaaggccttcaatgtaaacctcatcgacctcgcgacaaagtaagaagaccaagggtattccacacgcgcctcaagttcccttttcaacacactcttcgacacacatctccgaataaacataatctgcgtgcatgtttctctagacaccgagagatctacacacgcgcatatgtatatacatatgtatatatatatatatatatacttacatatatacatacgtatgtgtgcgtatgattccactagaggcaaagctaccggtagggaccgattttgaggtggatttgtttcgttctttctcttcgtttccttgtcgtttcgtctccagggtgatgattggcgcaccggtcactccgttgccgattcacttgatggacctttccttcgtctgcgtgaaagttccagttttctctttcgcgcgtcttcgcggctgcgacccggtccttggcgtggaaatgcggtcgactggagaagtaagcaggctgctgaagaaggagacgctattccgttttcaagtcgggaagcctggcgtcgtttgaggcagatctgcattgccgtctactcggcttggaacgatagacagaggaagacacaagtgcgaggaaggggaaagggaggaaaacagcgaaagaggaggaaagaagagtacggcacaatgtgcgagcgaagcgagaagtgagtgagagtgtttagaagaacaaagtgagaaacgaaaatgaaacttctgggtatccttccctcaagcgactgttcgtggacaaacgcagttttcactcgagacgtcaacgcgttccgtctccattgcttgtctcctcgcgaccgtgttgcttttcctttcacaggttgcgtgcttcggagccagcaaacatgaggctttcctcaaggctctcatctcggctggtgtgccgctgcctcttgagaagcgaacgattctcatcagcgcaggtacgcaacgttctgtaaactaagcgattctttctgttcgctttctctctgtcgagcgacagcaaccttttcctgtctccttccctccctctcgagacagagcagcttccatgttccccttagtgttttttaacagccgcgtttctctgaagtcggcgcatgcatctactattgcagatttcgcttcgtgttcgtgcgagccgaagaaatgctgcccgtcgccctggcccttcgactctctctcctcgtcagtttcgctctctccatttccagctggcgaagagtttctatggacatatgtctgtcggtcgcgtgtgatgcatgcactgcctctcggttgtgacattttatcctcttgtctttgcaggccctctgtggtcgaagatggaactcgagccgtacttcaaaatccttttggacctgggcttcacaatctacgcaacggaaggtaagtggaggcgcactccatgaatccctttgcagttctctcttctttttcttctcacactaacagttggtgttcctatctatctacctacctctctatctatgtctctctggttgtctgtctgcctctcggtccgtgtatctgtgtatctgtcggtgcagctctctaagccgcgaacggtcgtgcgtatatatatgttcatctgcaacacaacgaacgctctgtatgcacgtgccatgtcgatatgactgtgtacgctggccgtcgttgtctgtgtgcaggtgtattgcttcgccgctgcagaacgtagcatatgtgtctttattcaacatttttgaagggatattgccttcggtttctttcagtggcatttagctaggattttttccttttttttgaaaggtagtgaagaacgtgtgttgacggccagggccgcagcacatgctgcgatgtctttcttttgcccatggtgtgtgtgtgtgtgtcttgtgtggtcgctttggcttttcttcattggactgtttttcgttttgtgtttcttccatttttccgttcttcaagtgcatgcgaaggttgtcaagcgtattctctcggaattggctttcacaaagctcttctgccccgctcctcgtttgtttccacctttcggcttctttttgttcaacgttcgcatgctcacacgcatgcacgcctcgatcttttcctgtcctgacgcagacaagtgctcttcgttttactgctgtccccgtttccgctgccccgccgtgacttctctcgttttgcgagttcacgtttcttctttcttcgtttttctggattttcttcggtgcccaggtacctacagattcctcatgaacagcgtcgttcgcgggcaggggacccacctgcctgggaacgcgtcgccggcgtccgacagcggccttcggactcctacgacagccgagtccgacgcagatgcgtgcattcgcgcgaaatacgcatcgcgcattattcgcgtgagaaagccgattgtcggatcgaatgagtcgcacaacggaggtcaccagtcacctcacgctctctccctcattgaaagtggtaaggcccggcagtgcgttttcgggtggctgtgcagacggggcatgcgattttttgcgtttctgagcaacgaggcgccagcatgtacacacagcccagtggtttatgtcttgtttgctttcgtgttgcagtcgcacatgcatgcactcttatgtgtatgcgcatctatcgtgccaaggtccattcacatgcctagttatgtgcatgcggaggttgagatgcatactgaggatggtcgatgtttaaacgattgttgacaggctcagctattgggagcgagcatggtttttctctgtgagagttcactggaccctagaccgtaaacacttgcagagactggctggaccccctcgatcgaaaccatgcatgcagcagtcaagtcgaccacagacagaaacagacgcacagcagtgccacggagatgtaggtgcggcgttcacggaggtagaggtgaacatgtggtttcacatggctttttgaattttttcgcaagaaggaaagtacgagaacttcctgctatgcacgaatcgctcatcctttcttgccgaagaacgaaacggtgtcttgtttgttcacatagggaaggtcgaaatggtcatcaatgtgcctgacagcatgaaccaccgagcgggcacaaacggctacctgatgcgtcgcactgcgaccgactgcggaggtgcgttttgcctcagtgggcgcaacagacgaaccaaagaaacgaaagaaaagacagaaaaaaatggtggaactcgtgctctagcaaaacggactacccgacggaactgcaaagcgtctgtctggtccgagggcgtcttgccgttcccgactgggcgttcgaaaaaagcaagtcttctccatttcatgttttggcggcccgcgcaagcaacagtaatcttcactggcttggcccaccgactctcacgacctcagttcagtatgcgggcgcggggaatcaagtgcgaaatactcgttttctacataattatatatatatatatatatgtatatacgcgtacatagatataaatacggacatgtagacagatgtatgcatatgcatatatagttacaaacgtgtatagatttagacagattcgtaatattttgtgtatgtttcgatcaatacattaacgtttcgcttgcaatcagatgcgtcgatggcctcaacagtcgacagagcatggggtacgctgttttctggggtcgggaacgtttcagaaatctcgtcagagagagcgggcttctccgcgccattggcgtttgcgtgtctgcaatatgttgatcggcgtttacgtcgttttcttgtttttctcttcgcagttcccctcctgacaaacgtcaaagtggcaagcatgttcgtcgaggccctcaacaagaaagaagcgaaagaagctcagggtcgctccttctgggacattcgcagctgggatgaatactggcctcaaaaataa

SEQ ID NO: 2 - DNA, 5064 bp, Toxoplasma gondii
atgcctcacagtggagggcggagagctgttgctcccatttaccctctcgatctggcaggacgtctgagacctgcaatgctggtgttagccgatgggactgagtttctcggatactccttcggctacccaggcagtgtgggaggcgaggtcgttttcaacactggtatggtcggctaccccgagtctctgacggatccttcgtacgaggggcagatcctcgttctcacataccctctcatcggcaactatggcgttccctcttcggaaaaagatgagcatggcctgccgaaatactttgagggcgaccgcatttacgttcgcgctctcgttgtggcggactacgacaacgcagccgtgacggcacactttcgtgcagagaacagcctcagtgcttggatgaacactcacaaagtcccggcgattgcaggagtcgacacgcgagcgttgaccaagcacctgcgcgaggtcggctgcatgctgggcaagatcgtcgtcctgagcgaagaagaagagcgtcgatccggcttgtcgctctcggctctcgccgcgcttccctcagcgactgcagcagagcaacgaggagagaacgacgcgacggtgacgcccgacaaagcagaggcccgcctaagagtggagaggcgacaagcagcgctcacgatgtgggaggaggcgatccgcaacaaggcgaagaacctgccatgggaagaccccaacaaagacaacctcgtcgccctcgtttcgcgaaaagaagtgcgcgtgtacaaatctactgtcgtggatccgaatctccgcgacgtcctcatcctctgcgtggactgcgggatgaaatacaacatctaccgccagcttctccatagcaaattcgagcactgcaacatcattctcaaggtggtgccgtgggatttcgactttggcaacgacgaatttgacgggctcttcatcagcaacggtccaggcgaccctgagagatgcgaaaaaacagttgctaacattcgacgcgtcatggagcgaaagatccccatcttcggcatctgcctaggaaaccaacttcttgccctggctgccggcgcgagaacgtacaaaatgaaatacggaaacagaggaatgaatcagccggtgatcgacttgcgaacgtcgagatgctacatcacaccccagaaccatggctttgccgtcgacgagagcacgctgcctcgagatttcctgccgctctttgtaaatgcaaacgaccgttcgaacgaaggcatcatccatcgcacgctgccttttttctctgcacagttccacccagaggcgtcaggtggtccgacagacacgttttacttatttggcgacttcatcgcctcgattatgaaggcgcagacgctgaagcaggtccacacgactccgttctcctttccgcagaagttccagaaagttctgcttctcgggagcggaggcctgagcatcggccaagccggggagttcgactacagcggctctcaggcgataaaggcgctgaaagagcagaacatctttgtcgtcgtggtgaaccctaacatcgccacggtgcagaccagccagcacatggccgaccgggtgtacttcctgcccgtcacggatgagttcgtgacgaaagtcatcgaaaaggaaatgcccgacggcattctctgcacattcggaggccagacggccctcaactgcgctgtgaaactccacgaacaaggcgtcctggcaaaattcggctgcaaaatcctcggcagtccaatcgaagcgatcattgcgactgaggatcgaaaggtgtttgcggcgaagctggaagaaatcggagaaaaagtggcggagagcgcggccgcgacaaacacggaagaagctgtgcaagcggcgaaggccattggctaccccgtcctcattcgcgccgccttcgcactcggtgggctcggatctgggttcgccgaggacgaggagaccgtccgacgcatttgcaaggaagccttctcccattcttctcaggttttcgtggacaaaagcctgaagggctggaaggaggtggagtacgaagtcgttcgcgactgcaagaacaactgcatcaccgtctgcaacatggagaacttggatcccctcggcatccacacgggagattcgattgtcgttgctccttcgcagacgctgtctaacgaggactactaccgcctgagagataccgcgctgaaggtgattcgtcacttcggcatcgtcggcgaatgcaacatccaatacgcgctcgaccctaactcggagaaatactacatcgtcgaagtcaacgcgcgtctctctcggagcagcgccctcgcgtccaaagccactggctaccctttggcctacattgcggcgaagctcgctctcggctcgactttggtggagctcagtaactccgtgacaaaagagacaaccgcctgcttcgagccttcgctggattacgtcgtcaccaaggtgccgaggtgggatctgcgaaaattcgagtcgtgcgaccccctgatgggcagcgcgatgaagagtgtcggcgaggtcatggccattggacgcaccttcgaggagtctctgcaaaaggcgctcagaatggtggacgagaaggccggcggcttcgacgagtcggtctgtcactttttttccacggacgaggactgcgcgccttcgctgcccgggtcagacttcaagacgtcctcctctggagaatgcatgcgtggcggctgcggacgcacagactccggcgccgagcggcaggctgcgctgctggaggcggaacttcgccgtccgtcaccgaatcgaatctgggcgctggcgctcgccttccagctcgggtggacggtcgacgcgctccacgagaaaacgaaaatcgacaaatggtttttaagcaaacttcaaaacatcaacgacatcaagcgacagctcacccagctcaccctcgatgacctcacgcgcgcagatttcttctacatcaagaaatacggattcagcgaccgtcagattgcgcagtacttgatgaattcaccgagcgcggctgcgctgtcgcagttcgacgtgcgtcgtcggcgactgcacctgggcgtcagaccgtcggtgaagcaaatcgacactctcgcggccgagtttcctgctcacacgaattacctttacttgacctaccaaggaatcgacgacgacgtctcgcctctcgccgccacgccgtccgtctcggcggtcttcgctggcgcgcgagccgagaagagagaagaagaaaacgcagagacatgcagagacgacgaggacgaaagtctcctccgccgcctgagcaaaagctccagcgcgcggcttagaaccggcgaaggcgacgcacccggaaaacaatgttttgtggttctaggctgcggctgctaccgcataggatccagcgtcgagttcgattggtccgctgtttcgtgtgtccgtacacttcgctctcttggccaccacgcgattgtggtaaactgcaatcccgagactgtgtcgacggactacgacgtgagcgatcggctgtatttcgaagacttgagcttggaaacagtcttgaacatctgggacattgaagccccggcgggagtgattatttccgtgggcggccagacaccaaacacgctgtgctctgcactggagaagcagggcgtgaggatcgtcgggacgagtgtggcggcgatcgactgctgcgaggacagacacaagttctcccggctctgcgacgagctgaacatcgaccagccgaggtggaaggagttcaccgaccttcgcacagcgaaggccttctgccaagaggtcggctacccagtcctcgtgcgtccttcctacgttttgagtggtgctgccatgcgagtggtgaccgacgacgagcagctcgacgccttcctcaaaatcgcagctgtcgtcagtggcgaatctcccgtggtcatctccaagttcgtcgagaacgccaaggaagtggaatttgacagcgtcgcttgccggggggaaatcgtaaactttgcaataagcgagcatgtggagaacgcgggtactcactctggagacgcgactttgattctccctggacagaagctttacgtggaaacgattcgtcgcgtgaagaagatctcgcagaaactcgcgcgcgcactccaagtctcaggtccgttcaacatccagttcatctgcaaacagaacgacgtgaaagtcattgagtgcaacttgcgagcgtcgcgtactttccccttcatcagcaaggccttcaatgtaaacctcatcgacctcgcgacaaaggtgatgattggcgcaccggtcactccgttgccgattcacttgatggacctttccttcgtctgcgtgaaagttccagttttctctttcgcgcgtcttcgcggctgcgacccggtccttggcgtggaaatgcggtcgactggagaagttgcgtgcttcggagccagcaaacatgaggctttcctcaaggctctcatctcggctggtgtgccgctgcctcttgagaagcgaacgattctcatcagcgcaggccctctgtggtcgaagatggaactcgagccgtacttcaaaatccttttggacctgggcttcacaatctacgcaacggaaggtacctacagattcctcatgaacagcgtcgttcgcgggcaggggacccacctgcctgggaacgcgtcgccggcgtccgacagcggccttcggactcctacgacagccgagtccgacgcagatgcgtgcattcgcgcgaaatacgcatcgcgcattattcgcgtgagaaagccgattgtcggatcgaatgagtcgcacaacggaggtcaccagtcacctcacgctctctccctcattgaaagtgggaaggtcgaaatggtcatcaatgtgcctgacagcatgaaccaccgagcgggcacaaacggctacctgatgcgtcgcactgcgaccgactgcggagttcccctcctgacaaacgtcaaagtggcaagcatgttcgtcgaggccctcaacaagaaagaagcgaaagaagctcagggtcgctccttctgggacattcgcagctgggatgaatactggcctcaaaaataa

SEQ ID NO: 3 - protein, 1687 aa, Toxoplasma gondii
MPHSGGRRAVAPIYPLDLAGRLRPAMLVLADGTEFLGYSFGYPGSVGGEVVFNTGMVGYPESLTDPSYEGQILVLTYPLIGNYGVPSSEKDEHGLPKYFEGDRIYVRALVVADYDNAAVTAHFRAENSLSAWMNTHKVPAIAGVDTRALTKHLREVGCMLGKIVVLSEEEERRSGLSLSALAALPSATAAEQRGENDATVTPDKAEARLRVERRQAALTMWEEAIRNKAKNLPWEDPNKDNLVALVSRKEVRVYKSTVVDPNLRDVLILCVDCGMKYNIYRQLLHSKFEHCNIILKVVPWDFDFGNDEFDGLFISNGPGDPERCEKTVANIRRVMERKIPIFGICLGNQLLALAAGARTYKMKYGNRGMNQPVIDLRTSRCYITPQNHGFAVDESTLPRDFLPLFVNANDRSNEGIIHRTLPFFSAQFHPEASGGPTDTFYLFGDFIASIMKAQTLKQVHTTPFSFPQKFQKVLLLGSGGLSIGQAGEFDYSGSQAIKALKEQNIFVVVVNPNIATVQTSQHMADRVYFLPVTDEFVTKVIEKEMPDGILCTFGGQTALNCAVKLHEQGVLAKFGCKILGSPIEAIIATEDRKVFAAKLEEIGEKVAESAAATNTEEAVQAAKAIGYPVLIRAAFALGGLGSGFAEDEETVRRICKEAFSHSSQVFVDKSLKGWKEVEYEVVRDCKNNCITVCNMENLDPLGIHTGDSIVVAPSQTLSNEDYYRLRDTALKVIRHFGIVGECNIQYALDPNSEKYYIVEVNARLSRSSALASKATGYPLAYIAAKLALGSTLVELSNSVTKETTACFEPSLDYVVTKVPRWDLRKFESCDPLMGSAMKSVGEVMAIGRTFEESLQKALRMVDEKAGGFDESVCHFFSTDEDCAPSLPGSDFKTSSSGECMRGGCGRTDSGAERQAALLEAELRRPSPNRIWALALAFQLGWTVDALHEKTKIDKWFLSKLQNINDIKRQLTQLTLDDLTRADFFYIKKYGFSDRQIAQYLMNSPSAAALSQFDVRRRRLHLGVRPSVKQIDTLAAEFPAHTNYLYLTYQGIDDDVSPLAATPSVSAVFAGARAEKREEENAETCRDDEDESLLRRLSKSSSARLRTGEGDAPGKQCFVVLGCGCYRIGSSVEFDWSAVSCVRTLRSLGHHAIVVNCNPETVSTDYDVSDRLYFEDLSLETVLNIWDIEAPAGVIISVGGQTPNTLCSALEKQGVRIVGTSVAAIDCCEDRHKFSRLCDELNIDQPRWKEFTDLRTAKAFCQEVGYPVLVRPSYVLSGAAMRVVTDDEQLDAFLKIAAVVSGESPVVISKFVENAKEVEFDSVACRGEIVNFAISEHVENAGTHSGDATLILPGQKLYVETIRRVKKISQKLARALQVSGPFNIQFICKQNDVKVIECNLRASRTFPFISKAFNVNLIDLATKVMIGAPVTPLPIHLMDLSFVCVKVPVFSFARLRGCDPVLGVEMRSTGEVACFGASKHEAFLKALISAGVPLPLEKRTILISAGPLWSKMELEPYFKILLDLGFTIYATEGTYRFLMNSVVRGQGTHLPGNASPASDSGLRTPTTAESDADACIRAKYASRIIRVRKPIVGSNESHNGGHQSPHALSLIESGKVEMVINVPDSMNHRAGTNGYLMRRTATDCGVPLLTNVKVASMFVEALNKKEAKEAQGRSFWDIRSWDEYWPQK

SEQ ID NO: 4 - DNA, 24 bp, Artificial Sequence
  Degenerate Oligonucleotide Primer.
ccnytnggnathcayacnggngay

SEQ ID NO: 5 - DNA, 30 bp, Artificial Sequence
  Degenerate Oligonucleotide Primer.
ytcytcmaangtyctncckatngacatnac

SEQ ID NO: 6 - protein, 10 aa, Artificial Sequence
  Conserved amino acid sequence of CPSII domain.
PLGIHTGDSI

SEQ ID NO: 7 - protein, 12 aa, Artificial Sequence
  Conserved amino acid sequence of CPSII domain.
GEVMSIGRTFEE

SEQ ID NO: 8 - DNA, 33 bp, Artificial Sequence
  Synthetic Oligonucleotide Primer.
gggagatctatggcttcgtaccccggccatcaa

SEQ ID NO: 9 - DNA, 32 bp, Artificial Sequence
  Synthetic Oligonucleotide Primer.
ggggatcctcagttagcctcccccatctcccg

(19) United States
(12) Patent Application Publication
(10) Pub. No.: US 2002/0164754 A1
(43) Pub. Date: Nov. 7, 2002

(54) Compositions and methods for treatment of Toxoplasma gondii and other apicomplexans

(76) Inventors: David J. Bzik, Grantham, NH (US); Barbara Fox, Grantham, NH (US)

Correspondence Address: Licata & Tyrrell P.C., 66 E. Main Street, Marlton, NJ, 08053, US

(21) Appl. No.: 10/094,679

(22) Filed: Mar. 8, 2002

Related U.S. Application Data
(63) Continuation-in-part of application No. PCT/US01/03906, filed on Feb. 7, 2001.
(60) Provisional application No. 60/180,604, filed on Feb. 7, 2000.

(51) Int. Cl. C12Q 1/44; A61K 39/02; C12N 9/16

(52) U.S. Cl. 435/196; 435/19; 424/190.1

(57) ABSTRACT

Pyrimidine auxotroph mutants of apicomplexans are provided which are mutated in one of six enzymes of the de novo pyrimidine biosynthesis pathway. Also provided are methods of protecting an animal against infection by apicomplexans by administering a pyrimidine auxotroph mutant and methods for screening for inhibitors of pyrimidine salvage enzymes in apicomplexans.

### INTRODUCTION

This application is a continuation-in-part of PCT/US01/03906, filed Feb. 7, 2001, which claims the benefit of priority of U.S. Provisional Application No. 60/180,604, filed Feb. 7, 2000, now abandoned.

This invention was supported in part by funds from the U.S. government (NIH Grant No. R01 AI41930) and the U.S. government may therefore have certain rights in the invention.

### BACKGROUND OF THE INVENTION

Toxoplasma gondii is an obligate intracellular parasite which can infect many warm-blooded vertebrates including both mammals and birds. In humans, it has been recognized as a major cause of severe congenital disease and a common cause of infection in immunocompromised hosts. Recently, the parasite has received increased attention as an important opportunistic pathogen affecting up to 25% of AIDS patients (Kasper, L. H. (1994) Toxoplasma infection and toxoplasmosis. Harrison's textbook of Internal Medicine Ed. EiCE Braunwald, NY, McGraw-Hill, 13th edition 903-908). Improved chemotherapy for T. gondii is urgently needed to treat infected immunocompromised subjects.

The subclass Coccidiasina includes the order Hemosporidia, which contains the genus Plasmodium (causative agent of malaria). Coccidiasina also includes the order Eucoccidiorida which includes the suborder Eimeriorina. T. gondii belongs to the order Eucoccidiorida and to the suborder Eimeriorina. Within this latter order genera such Toxoplasma, Sarcocystis, Neospora, Eimeria, Cryptosporidium, Besnoitia and Hammondia are included.

T. gondii is relatively easy to handle and maintain. Consequently this parasite has become an important model for the study of how obligate intracellular parasites in the subclass Coccidiasina function.

It is known that mammals can be immunized against toxoplasmosis. However, all parasite strains and mutants so far developed as potential vaccines for toxoplasmosis share one common and serious flaw. Specifically, the T. gondii vaccines described thus far invariably kill immunocompromised animals, even with only administration of small parasite doses (Pfefferkorn, E. R. and L. C. Pfefferkorn. 1976. Exp. Parisitol. 39:365; Radke, J. R. amd M. W. White. 1999. Immunity 67:5292).

The present invention relates to a method for chemotherapy via creation of attenuated pyrimidine auxotroph mutants of obligate intracellular parasites of the phylum apicomplexa. A vaccine has now been developed for immunizing animals of various types against T. gondii. This vaccine makes use of a specific pyrimidine auxotroph mutant of T. gondii which has been found to give immunity without apparent concomitant chronic infection of the animal. It is believed that pyrimidine auxotrophic mutants can also be used to immunize animals against other apicomplexans.

### SUMMARY OF THE INVENTION

An object of the present invention is to provide nucleic acid sequences encoding carbamoyl phosphate synthase (CPSII) of T. gondii.

Another object of the present invention is to provide a pyrimidine auxotroph mutant of T. gondii which can be used as a vaccine against T. gondii infection. This live-attenuated pyrimidine auxotroph mutant of T. gondii described herein does not kill immunocompromised animals even when administered in high doses.

Another object of the present invention is to provide a method for protecting an animal against infection by T. gondii which comprises administering to an animal a pyrimidine auxotroph mutant of T. gondii which is mutated in one of the six enzymes of the de novo pyrimidine biosynthesis pathway.

Yet another object of the present invention is to provide pyrimidine auxotroph mutants of other apicomplexans and methods of using these mutants to protect against infection from other apicomplexans, wherein the mutants are mutated in one of the six enzymes of the de novo pyrimidine biosynthesis pathway.

The pyrimidine auxotroph mutants of the present invention can also be used to screen for novel inhibitors of pyrimidine salvage enzymes in T. gondii and other apicomplexans.

Another object of the present invention is a vaccine for protection against infection by an apicomplexan which comprises the pyrimidine auxotroph mutant of the present invention and a pharmaceutically acceptable carrier or diluent.

### BRIEF DESCRIPTION OF THE DRAWINGS

FIG. 1 shows the genomic DNA (SEQ ID NO:1) and cDNA (SEQ ID NO:2) derived clones obtained from the cpsII locus of T. gondii. Genomic DNA clones and their names are shown in [symbol] boxes, cDNA clones are shown in [symbol] boxes. The complete T. gondii CPSII cDNA is encoded on 37 exons spanning about 24 kb of the genomic DNA.

### DETAILED DESCRIPTION OF THE INVENTION

T. gondii has a complete pathway for the de novo biosynthesis of pyrimidines (Schwartzmann J. D. and Pfefferkorn, E. R. J. Parasitol. 1981 67:150-158; Asai et al. Mol. Biochem. Parasitol. 1983 7:89-100). UMP, the first major end-product of the pathway, is synthesized from bicarbonate, glutamine, ATP, aspartate, and phosphoribosyl pyrophosphate (P-rib-PP) is catalyzed by six major enzymes: carbamoyl phosphate synthase (CPS), aspartate transcarbamylase (ATC), dihydroorotase (DHO), dihydroorotase dehydrogenase (DHOD), orotate phosphoribosyl transferase (OPT), and orotidylate decarboxylase (ODC or URA3). The pathway begins with CPS which combines glutamine, ATP and bicarbonate to form carbamoyl phosphate. The glutamine-specific CPS activity involved in de novo pyrimidine biosynthesis is referred to as CPSII and the enzyme is typically localized in the nucleolus of eukaryotic cells (Davis, R. H. Microbiol. Reviews. 1986 50:280-313). ATC then combines carbamoyl phosphate and aspartate to form carbamoyl aspartate. The third reaction, catalyzed by DHO, yields dihydroorotate. DHOD then oxidizes dihydroorotate to orotate with the reduction of NAD. OPT then phosphoribosylates orotate to OMP. The sixth step, catalyzed by OMP Decarboxylase (URA3), converts OMP to UMP. UMP is the precursor of all pyrimidine nucleotides and deoxyribonucleotides.

In the Urea Cycle of ureotelic animals, carbamoyl phosphate is combined with ornithine, derived from ammonia, to form citrulline during de novo arginine biosynthesis. The CPS involved in arginine biosynthesis is referred to as CPSI. In some eukaryotes such as yeast, where CPSI is cytosolic, mutants of CPSII are a bit leaky because of some “mixing” of these two pools of carbamoyl phosphate. In many eukaryotes, CPSI is confined to the mitochondrial matrix and carbamoyl phosphate produced from CPSI in the Urea Cycle is unavailable to the carbamoyl phosphate “pool” which feeds into de novo pyrimidine biosynthesis (Davis, R. H. Microbiol. Reviews. 1986 50:280-313). There is no mixing of CPSI and CPSII in T. gondii due to either sequestering of CPSI to a compartment such as the mitochondria or a lack of CPSI type activity in T. gondii. Thus, the CPSII involved in the de novo biosynthesis of pyrimidines is the first committed step of the de novo pathway of pyrimidine synthesis in T. gondii.

Comparative studies across many genera demonstrate extensive diversity in the de novo pathway's regulatory mechanisms, in the structure of its enzymes, and in the organization of the genes which encode the enzymes (Jones, M. E. Ann. Rev. Biochem. 1980 49:253-279). In many organisms, including man, the first three enzymes of de novo pyrimidine biosynthesis are found as multifunctional polypeptides. Typically, in higher eukaryotes the CPS, ATC, and DHO activities are encoded on a single gene, CAD, that specifies a single multifunctional polypeptide chain. In lower eukaryotes such as S. cerevisiae, the CAD-homologue gene specifies functional CPS and ATC domains, but a non-functional DHO domain. The organization of these CAD activities has evolved differently in various parasitic protozoa. In protozoan parasites of phylum Apicomplexa, including Babesia and Plasmodium species, the CPS activity is specified as an individual gene specifying a polypeptide with a single CPSII enzyme activity comprising the glutamine amido transferase (GAT) activity and the CPS activity; GAT+CPS=CPSH (Chansiri K. and Bagnara, A. S. Mol. Biochem. Parasitol. 1995 74:239243; Flores et al. Mol. Biochem. Parasitol. 1994 68: 315-318). This peculiar protozoan parasite gene organization is more similar to bacteria where CPS is monofunctional (Mergeay et al. Mol. Gen. Genet. 1974 133: 299-316.). T. gondii is also an Apicomplexan and it also specifies the CAD enzyme activities on individual polypeptides (Asai et al. Mol. Biochem. Parasitol. 1983 7:89-100). This difference in CAD gene organization between man and Apicomplexan parasites is reminiscent of the situation with DHFR and TS where these enzyme activities are present on a single polypeptide in Apicomplexan parasites (Bzik et al. Proc. Natl. Acad. Sci. USA 1987 84:8360-8364) and on individual polypeptides in man. The difference in DHFR-TS gene structure between parasites and man has provided significant opportunity for chemotherapy using compounds such as pyrimethamine.

The difference in the CAD gene structure for pyrimidine synthesis between parasites and man also provides a unique chemotherapeutic opportunity. Further, blocking the accumulation of UMP by attacking one of the de novo pyrimidine biosynthetic enzymes should have a more profound anti-parasite effect than, for example, blocking accumulation of dTMP via pyrimethamine and sulfonamide treatment which is the standard chemotherapy for recrudescent toxoplasmosis. The latter strategy primarily blocks tachyzoite DNA replication with little apparent effect on bradyzoites, whereas the former strategy is predicted to block both parasite RNA synthesis as well as DNA replication.

In addition to the novel protozoan gene organization of CAD, the CAD-encoded enzymes have unique properties and regulation that make them attractive targets for chemotherapy. The CPSII activity detected in T. gondii is primarily involved in de novo pyrimidine biosynthesis based on substrate preference (Asai et al. Mol. Biochem. Parasitol. 1983 7: 89-100). While the mammalian CPSI involved in the Urea Cycle is activated by N-acetyl glutamate, the CPSII activity found in T. gondii is not affected by this treatment. The T. gondii CPSII activity is inhibited by UTP, suggesting a pyrimidine-controlled regulatory circuit. While the CPSII activity of man is activated by P-rib-PP, the T. gondii CPS activity is not. In contrast to CPSII, other enzymes of the de novo biosynthetic pathway were broadly characterized to behave similarly to their higher eukaryotic counterpart. The T. gondii CPSII appears to have markedly different properties from mammalian CPSII (Asai et al. Mol. Biochem. Parasitol. 1983 7:89-100).

While T. gondii has a complete system for de novo pyrimidine biosynthesis, it only has a limited capacity to salvage pyrimidine bases. A biochemical survey of pyrimidine salvage enzymes supports the theory that all T. gondii pyrimidine salvage is funneled through uracil (Iltzsch, M. H. J. Euk. Micro. 1993 40:24-28). T. gondii has only three enzymes that are involved in salvage of pyrimidine nucleobases and nucleosides: cytidine/deoxycytidine deaminase, which deaminates cytidine and deoxycytidine; uridine phosphorylase, which catalyzes the reversible phosphorolysis of uridine, deoxyuridine, and thymidine; and uracil phosphoribosyltransferase (UPRT), which catalyzes the formation of UMP from uracil. The uridine phosphorylase and UPRT activities are the key salvage enzymes since pyrimidine salvage funnels all pyrimidine compounds first to uracil and then the UPRT activity yields UMP (Pfefferkorn, E. R. Expt. Parasitol. 1978 44: 26-35; Iltzsch, M. H. J. Euk. Micro. 1993 40: 24-28). The limited T. gondii pyrimidine salvage pathway is not required for viability. Mutations that abolish UPRT activity are tolerated and are equally viable to wild-type parasites in vitro and in vivo (Pfefferkorn, E. R. Expt. Parasitol. 1978 44:26-35; Donald, R. G. K. and Roos, D. S. Proc. Natl. Acad. Sci. USA. 1995 92:5749-5753). Furthermore, there is no available evidence that T. gondii actually salvages any pyrimidine bases from the host cell under normal in vivo or in vitro growth conditions.

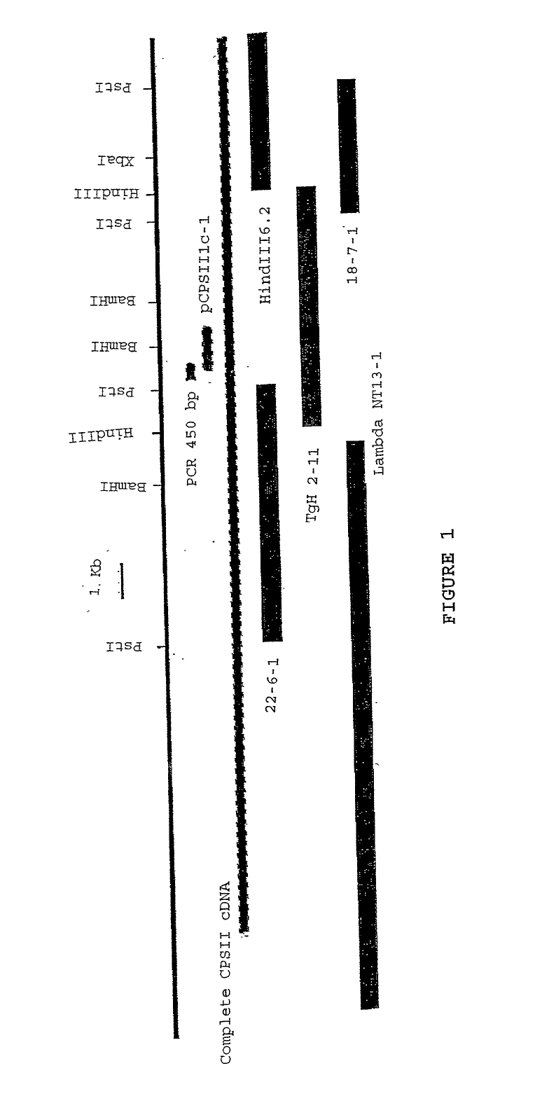

The DNA sequence of carbamoyl phosphate synthetase II (cpsII) has now been cloned. To clone the CPSII gene from the RH strain of T. gondii, a forward degenerate primer: (CCN)(C/TTN)(GGN)(ATA/T/C)(CAT/C)(ACN)(GGN)(GAT/C) (SEQ ID NO:4) and a reverse degenerate primer: (T/CTC) (T/CTC) (A/CAA) (NGT) (T/CCT) (NCC) (G/TAT) (NGA) (CAT) (NAC) (SEQ ID NO:5), were designed from two stretches of amino acid sequence (PLGIHTGDSI; SEQ ID NO:6) and (GEVMSIGRTFEE; SEQ ID NO:7), respectively, which were well-conserved in the CPSII domain from various species. With these two primers, PCR amplification of strain RH single stranded cDNA derived from RH mRNA was performed in accordance with known procedures (Fox et. al Mol. Biochem. Parasitol. 1999 98: 93-103). A PCR product of the expected length, PCR 450 bp (see FIG. 1) was obtained. The 450 bp amplicon was excised from agarose, purified and cloned into the SS phage vector M13 mp9 in both orientations, for single-stranded sequencing using the dideoxy termination method.

The purified amplicon was then reamplified and used to probe lamba phage cDNA libraries from the NIH AIDS Reference and Reagent Center and a 1.2 Kb cDNA phagemid clone (pCPSII lc-1) was identified and transduced to bluescript plasmid for analysis via the manufacturer Stratagene's protocol. A 1.0 Kb EcoRI fragment from pCPS lc-1 was shotgunned into M13 mp9 and SS dideoxy sequenced. The sequences were found to align to those of the original 450 bp M13 mp9 clone and to have high homology to CPSII of other species. Separately, Southern blots of T. gondii genomic restriction digests were probed with the gel purified 450 bp fragment. This probe hybridized to several restriction fragments derived from RH parasite DNA including a unique band generated by HindIII (6.5 Kb) (see FIG. 1). Genomic libraries were then constructed in bluescript SKII⁺ phagemid vector that would contain the 6.5 Kb HindIII fragment and these genomic libraries were screened with the labeled 450 bp PCR derived CPSII cDNA. Positive clones containing the desired insert in both orientations were isolated. The ends of TgH 2-11 6.5 Kb clone were then dideoxy double stranded sequenced using T3 and T7 primers. Primers from the ends of the sequenced sections were used to sequence the remainder. The 6.5 Kb HindIII fragment was also used to screen additional T. gondii genomic southerns and three PstI fragments were identified. Subsequently, a PstI T. gondii genomic library was constructed using standard methods and probed with fragments from either end of TgH 211 plasmid to yield 7.5 Kb, 4.9 Kb, and 3.8 Kb PstI clones that matched the corresponding sizes on the southern. Locations of these clones within the genomic DNA and cDNA of T. gondii are depicted in FIG. 1.

Collectively, the RH genomic DNA clones obtained specify T. gondii cpsII amino acids that have significant homology to cpsII amino acid 304-1400 of the T. cruzi encoded cpsII protein. Alignment of the derived amino acid sequence of T. gondii CPSII as set forth in SEQ ID NO:3 was highest with the corresponding sequences of CPSII from the parasitic organisms, P. falciparum, B. babesia, and T. cruzi, approximately 35 to 45% amino acid homology.

The remainder of the cpsII genomic DNA clones were sequenced using a “walking” primer approach (see FIG. 1). For example, fragments of DNA at the 5′ end of clone 22-6-1 and the 3′ end of clone 18-7-1 (see FIG. 1) were used to identify additional fragments on Southern blot to obtain appropriate clones encoding the full genomic cpsII coding region plus flanking regulatory sequences. The full length genomic DNA sequence of T. gondii cpsII is depicted in SEQ ID NO:1. The cDNA of T. gondii is depicted in SEQ ID NO:2.

Mutant T. gondii were also prepared wherein CPSII activity was knocked out. Knock out of this enzymatic activity or any other de novo pyrimidine synthesis enzyme was predicted to produce a pyrimidine auxotroph that would be attenuated in mammals due to the inability of mammalian cells to provide the abundant pyrimidines needed by the parasite for growth. However, salvaging the growth of T. gondii purely by feeding pyrimidine compounds to the parasite in the growth medium was unpredictable. Thus, pyrimidine salvage in T. gondii was examined.

Initial experiments primarily involved an enzymatic analysis of drug resistant mutants and the incorporation of various pyrimidine analogs into T. gondii RNA and DNA as an indication of pyrimidine salvage when parasites were grown in either normal host or mutant host cells. All biochemical communications between the parasite and host cells cross the vacuolar membrane which is now known to contain “pores” that permit the passage of nucleobases ranging in molecular size from 112 daltons up to 244 daltons. The size of the pores is estimated to be approximately 1500 daltons. A T. gondii mutant resistant to 5-fluorodeoxyuridine (FUDR-1) had lost uracil phosphoribosyltransferase (UPRT), an enzyme which is absent in normal host cells (Pfefferkorn, E. R. Expt. Parasitol. 1978 44:26-35). Labeling of wild-type parasites or FUDR-1 parasites with [³H]deoxyuridine, [A] uridine, and [³H]uracil revealed a striking pattern of pyrimidine incorporation into host or parasite nucleic acids. [³H]deoxyuridine was incorporated into wild-type T. gondii and labeled the host cell nucleus (DNA only since deoxyuridine is mainly converted into TTP by host cell enzymes). FUDR-1 mutant parasites were not labeled with [³H]deoxyuridine. [³H]uridine was incorporated into wild-type parasites and labeled host DNA (nucleus) and host RNA (cytoplasm) (uridine is incorporated into the host cell UTP pool by host cell UTP pool by host enzymes). FUDR-1 mutant parasites were not labeled with [³H]uridine. [³H]uracil was incorporated into wild-type T. gondii and did not label either host DNA or RNA. FUDR-1 (UPRT knock-out) parasites were not labeled with [³H]uracil.

In addition to the labeling patterns observed, wild-type RH parasites did not incorporate labeled orotic acid, orotate, cytosine, cytidine, thymine, or thymidine nucleobases. These results suggest that none of the host pyrimidine nucleotide pool is available to the parasite. Similarly, since uracil only labels the parasite, due to the parasite UPRT which is absent in the host, the pyrimidine pools of the parasite are also unavailable to the host. Thus, there is no detectable pyrimidine traffic detected between the parasite and host.

Experiments to evaluate the feasibility of constructing pyrimidine auxotrophs of T. gondii were performed. Pyrimidine auxotrophy relies on the ability to feed mutant parasites a pyrimidine nucleobase, such as uracil, in culture medium in amounts that will restore parasite growth to near normal levels. Experiments were therefore conducted to measure whether uracil incorporation into parasites in culture could account for normal replication and normal growth rates. In these experiments, biochemically saturating amounts of [³H]uracil (25 μg/ml) were added to the growth medium and the quantitative incorporation of label into parasite RNA and DNA was determined over a four hour interval. It was calculated using known values that about 82% of the pyrimidines incorporated into parasite nucleic acids were derived from the uracil which was added to the growth medium as a supplement (Table 1).

Table 1: Uracil Incorporation Assay Results and Calculation of Uracil Incorporation

Time: Parasites were labeled for 4 hours.

Culture: Contained 2.09×10⁷ parasites.

Medium: Contained [³H]uracil at 25 μg per ml or 0.223 μmole per ml.

Specific activity of uracil: 34.3 CPM per pmole (CPM=counts per minute).

Results: The parasite culture incorporated 2.27×10⁵ CPM in 4 hours.

Calculation:

1. Actual incorporation per parasite=0.11CPM per tachyzoite=0.00032 pmoles pyrimidine per tachyzoite per 4 hours.

2. Tachyzoites contain 0.10 pg DNA per cell.

3. Tachyzoites contain 0.50 pg nucleic acid per cell (The RNA:DNA ratio is 4:1).

4. Toxoplasma nucleic acids contain 112:760 pyrimidine by weight, thus: tachyzoites contain 0.50×0.1476=0.074 pg of pyrimidine

5. Pyrimidine molecular weight=112.

6. Tachyzoites contain 0.074 pg/112/pg/pmole=0.00066 pmoles pyrimidine in nucleic acid.

7. Tachyzoites double in 6 hours. This represents a 1.6-fold increase in the 4 hour labeling period, or equals the synthesis of 60% of nucleic acids in 4 hours.

8. Theoretical 100% incorporation=60% of 0.00066 pmoles=0.00039 pmole pyrimidine incorporated per tachyzoite per 4 hours.

9. Actual incorporation (0.00032 pmoles) therefore represents 82% of the theoretical maximum incorporation (0.00039 pmoles).

Conclusion: 82% of incorporated pyrimidines originate from uracil when it is added to medium.

An 82% efficiency of incorporation of exogenously added uracil into parasite nucleic acid was detected. However, the same pathway mediating this incorporation (UPRT) can be completely abolished with no effect on parasite growth rates in vitro or in vivo. These results indicate that the parasite may activate the uracil salvage pathway to obtain near normal levels of uracil directly from growth medium when it is available or when uracil is needed, or alternatively to completely rely upon de novo biosynthesis when uracil is unavailable extracellularly. These biochemical measurements supported the feasibility of constructing stable pyrimidine auxotrophs of T. gondii by constructing a knock out of a gene and enzyme activity of the de novo pathway.

A modified hit and run mutagenesis was devised for knocking out the T. gondii gene encoding CPSII. First, a new plasmid vector was developed for positive and negative selection analogous to the plasmid described by Fox et al. (Mol. Biochem. Parasitol. 1999 98: 93-103), except using the herpes simplex virus type I thymidine kinase (TK) gene instead of bacterial cytosine deaminase in the linker region of DHFR-TS. To create this plasmid two primers, a forward primer GGGAGATCTATGGCTTCGTACCCCGGCCATCAA (SEQ ID NO:8) and a reverse primer GGGGATCCTCAGTTAGCCTCCCCCATCTCCCG (SEQ ID NO:9) were used to PCR amplify via standard conditions the ganciclovir hypersensitive TK75 HSVTK allele (Black et al. Proc. Natl Acad. Sci. USA 1996 93: 3525-3529). The forward primer contains a BglII site and the reverse primer a BamHI site. Following BamHI and BglII digestion of the ˜1130 bp PCR product, the TK allele was ligated into plasmid pDHFRm2m3-FLG-TS which was digested at the unique BamHI site in the FLAG epitope linker. The TK PCR primers were designed to join with pDHFRm2m3-FLG-TS to produce an inframe insertion of TK between DHFR and TS in a plasmid called pDHFRm2m3-TK-TS, similar to previously described pDHFRm2m3-CD-TS plasmid (Fox et al. Mol. Biochem. Parasitol. 1999 98: 93-103). The trifunctional enzyme plasmid with TK was tested to confirm function of all three enzymes. Transfection of T. gondii with pDHFRm2m3-TK-TS and selection in 1 μM pyrimethamine produced parasites resistant to pyrimethamine. All subclones of T. gondii transfected with pDHFRm2m3-TK-TS (more than 100 clones of T. gondii) that are pyrimethamine resistant uniformly and concomitantly become sensitive to minute concentrations of ganciclovir. All T. gondii carrying a single allele of TK (or more than one allele) from pDHFRm2m3-TK-TS do not form plaques in 0.5 μM ganciclovir.

The TgH 2-11 clone of T. gondii CPSII was fused with the pDHFRm2m3-TK-TS plasmid to create a new plasmid suitable for modified hit and run mutagenesis. First, a 0.5 Kb segment of TgH 2-11 was removed from the 3′ end by digestion with BglII with BamHI. Resulting 2.6 and 1.2 kb DNA fragments were resolved in agarose and the 2.7 kb 3′ BamHI/BglII fragment was religated with the large DNA fragment from the same digest which contained the 5′ side of the HindIII fragment and the plasmid DNA. The correct orientation was mapped by restriction digestion. This created a modified HindIII fragment of CPSII gene with a central 1.2 Kb deletion between amino acids 723 and 1070 based on numbering from the T. cruzi CPSII gene. Finally, the trifunctional DHFR-TK-TS enzyme from pDHFRm2m3-TK-TS was added into the deleted HindIII (delta 1.2 Kb BamHI) plasmid by digestion of pDHFRm2m3-TK-TS with NheI and XbaI and ligation into the unique XbaI site of the deleted 1.2 Kb BamHI HindIII TgH 2-11 plasmid. A clone with inverted directionality of DHFR-TK-TS and CPSII expression was obtained and this plasmid was called p53KOX3-lR. The theory of targeting disruption of the endogenous T. gondii CPSI gene with p53KOX3-lR is that insertion of this plasmid by a single cross-over recombination would yield a C-terminal truncated CPSII with the 1.2 Kb BamHI deletion described above with a normal endogenous promoter but no untranslated 3′ regulatory region, and a duplicated cpsII allele with a N-terminal truncated (deleting everything before the HindII site at amino acid 663) CPSII and a normal non-translated 3′ regulatory region but no T. gondii promoter. Thus, single homologous crossover between truncated and deleted CPSII contained in p53KOX3-lR and the endogenous T. gondii locus would produce two mutant forms of CPSII with significant structural disruptions.

Wild type RH parasites were transfected with p53KOX3-lR and selected in the presence of 1 μM pyrimethamine and 200 μM uracil. Following lysis of the primary flask with p53KOX3-lR transfected parasites after 4 days of growth in 1 μM pyrimethamine plus 200 μM uracil, parasites were diluted 1:100 and inoculated into a second flask of fresh HFF cells under the same growth conditions. The second growth cycle is necessary for efficient selection of stable plasmid integration under pyrimethamine selection. Thus, transfected parasites must undergo approximately 25 cycles of replication prior to subcloning and screening for potential mutants. It is obvious that any mutant with any moderate or significant defect in growth rate would be quickly diluted in number by rapidly growing parasite in the mixed cultures. Following the second growth cycle of the primary transfection of p53KOX3-lR in pyrimethamine plus uracil medium, parasites were subcloned into a duet of 96 well trays with or without uracil supplementation. Individual wells were scored microscopically at 4-5 days post subcloning to mark wells with one viable parasite (a subclone) based on the presence of a single zone of parasite growth in that well. Typically 10 to 20 wells of a 96 well tray were successful subclones. Successful subclone wells were individually mixed by washing up and down with a 50 μl pipette to mix parasites to infect the whole HIFF monolayer in that well. Typically 3 further days of incubation produced total well lysis and many free infectious extracellular parasites. From these wells parasites were individually picked and inoculated (separate additions) into parallel wells of HFF cells in 96 well trays that contained the same growth medium (pyrimethamine plus uracil 200 μM) or trays only containing pyrimethamine 1 μM. If CPSII were knocked out in any of the T. gondii subclones a difference in growth rate could be detected by visual microscopic examination of identically inoculated wells. No less than 1 μl was tested at this point due to reliability of transfer and parasite number of inoculum. Thus, the concentration of residual uracil in the “no uracil” tray was actually still ˜1 μM. More than 800 subclones of T. gondii were eventually screened using the above assays, with more than 200 subclones being generated in each of four independent selections and transfection experiments with p53KOX3-lR. Following an initial assessment of growth rate estimate in the plus uracil or “minus uracil” condition, a number of putative clones were evaluated in a second test of uracil growth dependence. Following a second positive test of uracil growth dependence, a third test using 25 cm² HFF flask was performed under conditions of a uracil concentration less than 0.1 μM. From these selections four T. gondii mutants were obtained which had a quantitative assessment of at least a detectable growth dependency on addition of uracil to the growth medium. These were putative T. gondii uracil auxotrophs. One independent transfection produced mutant cps1, a second independent transfection produced mutant cps2, and a third independent transfection produced mutants cps3 and cps4. Each of these mutants was found to be highly sensitive to ganciclovir, loosing ability to form plaques in only 0.5 μM ganciclovir. The mutants were grown and genomic DNA was isolated from each mutant and wild type RH parasites from the contents of 2 or more 25 cm² flasks for each DNA isolation to document integration of targeting disruption plasmid p53KOX3-lR into the endogenous CPSII locus by homologous recombination. The plasmid p53KOX3-IR could form two general patterns of integration based on recombination either 5′ of the BamHI deletion, or recombination 3′ of the BamHI deletion. HindII digested cps1, cps2, cps3, cps4 and RH parasite genomic DNA was subjected to Southern blotting and hybridized to labeled gel purified 6.6 Kb HindIII fragment of TgH 2-11 encoding T. gondii CPSII sequences. A 5′ cpsII integration would produce at least fragment sizes of ˜6.5 Kb and 7.8 Kb following digestion with HindIII, whereas a 3′ cpsII integration site would produce at least fragments of 5.0 Kb and 7.8 Kb when digested with HindIII. If the plasmid were duplicated at the time of integration which is seen frequently with the pDHFRm2m3-TS plasmid backbone (Sullivan et al. Molecular and Biochemical Parasitol. 1999 103:1-14) then an additional fragment at 7.8 or 9.5 Kb could be generated by integration at endogenous CPSII. Each of the selected putative CPSII mutants had undergone an integration of plasmid p53KOX3-1R at either the 5′ location (cps1, cps2, cps3, or the 3′ location, cps4). Mutant cps4 had multiple bands between 7.8 and 9.5 Kb and additional bands at higher molecular weights suggesting integration of plasmid at CPSII and other loci. In contrast, mutants cps1, cps2 and cps3, obtained in independent transfections and selection, had identical patterns of hybridization to CPSII DNA suggesting that the targeting plasmid p53KOX3-IR only integrated into the 5′ site of the CPSII target region and each mutant had duplicated the plasmid DNA upon integration (the 9.5 Kb DNA band). Thus, successful targeting to and disruption of the T. gondii CPSII locus was demonstrated.

Each of the mutants (cps1, cps2, cps3, and cps4) have a phenotype of uracil growth dependence. However, all of these mutants are somewhat “leaky” in that there was not an absolute growth (replication) dependence on uracil addition to the growth medium for replication. Each of these mutants grows at a moderate (½ of normal) growth rate for the first 2 days following infection of a host cell producing vacuoles that contained 16 to 32 parasites by 3 days post infection. In contrast, RH parasites are lysed out of their primary vacuoles at this time (3 days, >64 parasites). However, the cps mutants slow after 3 days and many parasites never (about ⅓) break out of their primary vacuole. If the primary vacuole breaks, a few parasites can be detected at the site of infection but the infection site always involves a small zone of infection that never forms a visible plaque in a standard 7 day plaque assay. To quantitate growth of these mutants, HFF flasks were inoculated with cps1 or cps2 parasites (about a multiplicity of infection (MOI) of 1 parasite per 20 HFF cells). After 2 hours of attachment and invasion (all of these mutants have normal attachment and invasion phenotypes as scored by counting percent entered parasites into host cells as a function of time post-inoculation), different concentrations of different pyrimidine compound was added to parallel infected HFF flasks. As a function of time post pyrimidine addition (t=0 hour) the number of parasites per vacuole was scored for 50 vacuoles as described by Fox et al. (Mol. Biochem. Parasitol. 1999 98:93-103). The number of parasite doublings was calculated based on 1 parasite entering each primary vacuole. Thus, 1 doubling=2 parasites per vacuole, 3 doublings=8 parasites per vacuole, and 5 doublings=32 parasites per vacuole. The pyrimidine dependence of cps1 and cps2 replication (doublings of parasites in the vacuole) was plotted graphically. Relatively low concentrations of uracil, uridine, deoxyuridine, cytidine, and deoxycytidine completely rescued the growth rate of cps1 and cps2 mutants to wild type RH levels. This pattern of rescue is precisely consistent with the limited set of salvage enzymes available to T. gondii suggesting that these mutants have a defect in de novo pyrimidine synthesis that can be corrected by salvage of pyrimidines from exogenously supplied pyrimidines in growth medium in vitro. Cytosine, as expected, did not rescue at all. The response to thymine and thymidine was additionally informative about the cause of the growth defect in cps1 and cps2. Moderate concentrations of thymine or thymidine partially rescued the replication of cps1 and cps2, whereas very high concentrations of these pyrimidines did not rescue replication. This is believed to be caused by the putative defect in the cps1 and cps2 pyrimidine auxotrophs, that is a reduced “pool” size of UMP. If UMP pools (ultimately used for RNA and DNA synthesis) are lowered it follows that a resulting decrease in TMP pools is expected since UMP is the precursor of TMP in T. gondii and all other apicomplexan parasites which normally lack TK activity. However, since cps1 and cps2 now express a TK activity carried into the parasite by the p53KOX3-1R plasmid, exemplified by sensitivity of these mutants to ganciclovir (specific to HSV TK), feeding parasites either thymine or thymidine is expected to increase TMP pools. Thus, it appears that moderate levels of thymine or thymidine partially rescue growth of cps1 and cps2 by restoring TMP pools. These data indicate that there is indeed a defect in accumulation of UMP and TMP pools in the cps1 and cps2 mutants. The defect is most easily rescued by feeding the parasite pyrimidines that can be incorporated (uracil, uridine, deoxyuridine, cytidine, deoxycytidine) and can be partially rescued with thymine or thymidine (the TMP pool). Since the parasite has no mechanism to convert TMP back to UMP there is still a defect in the UMP pool in cps1 and cps2 even with added thymine or thymidine and rescue is never complete. The ability of cps1 and cps2 mutants to form plaques on HFF monolayers in the standard 7 day assay paralleled the pyrimidine dependence of parasite replication in vacuoles.

The cps1 and cps2 mutants were inoculated intraperitoneally (ip) into balb/c mice to measure parasite virulence compared to virulent RH parasites. Both mutant cps1 and cps2 had equal virulence as RH parasites in balb/c mice (Jackson Labs), killing all mice within 10 days of ip inoculation (group size was 4 mice per parasite strain). Only 100 parasites (˜50 PFU) of each parasite strain was needed to kill all mice in each group. This pattern of virulence can be understood by re-examining the pyrimidine dependence of cps1 and cps2 growth. Uridine is believed to be the pyrimidine responsible for virulence of cps1 and cps2 in mice. Only 5 μM uridine completely rescues normal plaque size of cps1 and cps2 in vitro. Plasma concentrations of uridine in mice are approximately 5 to 10 μM. Thus cps1 and cps2 grow normally in mice and are not attenuated at all.

It is believed that the single recombination into the CPSII locus only partially disrupted expression of CPSII activity in cps1 and cps2. Accordingly, cps1 and cps2 are not “complete” pyrimidine auxotrophs. Cps1 and cps2 were thus utilized as the parent strain background in which to select a more highly attenuated pyrimidine auxotroph mutant. Both the cps1 and cps2 mutants express a TK allele which was inserted into the CPSII locus. Hence, cps1 and cps2 mutants were grown for several generations in the absence of pyrimethamine and in the presence of 200 μM uracil. Then, approximately 1-2×10⁵ cps1 or cps2 parasites was inoculated into a 25 cm² HFF flask and selected negatively in the presence of 10 μM ganciclovir plus 200 μM uracil. This is 20 times the dose of ganciclovir necessary to completely block plaque formation of these mutants. After approximately 10 days, an outgrowth of viable parasites was observed for both the cps1 and cps2 selections. The parasites which were growing in ganciclovir plus uracil were subcloned in ganciclovir and uracil (same conditions) and individual clones, cps1-1 and cps2-1, were identified from each parent, respectively, for further analysis. The cps 1-1 and cps2-1 subclones were first tested for their sensitivity to pyrimethamine. The theory of negative selective in only ganciclovir is that a mutant that disrupts expression of the TK allele should simultaneously acquire sensitivity to pyrimethamine due to loss of the expression of the fused trifunctional DHFR-TK-TS transgene(s) inserted into the CPSII locus of cps1 and cps2. Indeed, loss of sensitivity to ganciclovir (10 μM) in cps1-1 and cps2-2 correlated perfectly with a gain of sensitivity to pyrimethamine (1 μM) in both replication and plaque assays.

Evaluation of the pyrimidine dependence of growth of cps1-1 and cps2-1 compared to the cps1 and cps2 parents, respectively, revealed that the newly selected mutants (cps 1-1 and cps2-1) were absolute pyrimidine auxotrophs. No pyrimidine compound at less than 25 μM could rescue plaque formation of cps1-1 or cps2-1. Further, only uracil and deoxyuridine provided significant growth rescue, and only in relatively high doses. Uridine was quite poor at rescue of plaque formation in cps1-1 and cps2-1. In pyrimidine concentrations up to 200 μM only uracil completely rescued plaque formation of cps1-1 and cps2-1. In addition, as expected from ganciclovir resistance (no TK expression) phenotype, no response was detected to thymine or thymidine.

A more detailed growth response to pyrimidine, measured as parasite replications (doublings) was performed for cps1-1 and cps2-1 and compared to the results previously obtained for cps1 and cps2. In the absence of added pyrimidines or the presence of even high concentrations of thymine, thymidine, deoxycytidine, cytidine, or cytosine, a cps1-1 or cps2-1 parasite that entered a vacuole in a host HFF cell remained as a single non-replicated parasite, not only in a 36 hour replication assay, but also upon continued incubation of infected cultures in vitro. Uridine rescue was poor, with a slow restoration of growth at very high amounts, >400 μM. Deoxyuridine rescue was significant, but again, full growth rate was not restored at any concentration of deoxyuridine. Rescue with uracil was robust, but only in a limited range of concentration. Full restoration of growth rate in cps1-1 and cps2-1 was possible only with uracil added between 200 and 400 μM. Lower concentrations of uracil rescued poorly and concentrations of uracil higher than ˜500 μM reduced the growth rate of cps1-1 and cps2-1 significantly. In fact, cps1-1 and cps2-1, as well as cps1 and cps2, do not even form plaques in 2000 or 4000 μM uracil, conditions with no effect on RH parasite plaques. This result is dichotomous and suggests that there is an intimate, regulatory loop in T. gondii pyrimidine salvage that is down regulated by very high concentrations of uracil. This phenotype can only be observed in T. gondii pyrimidine auxotroph mutants, not in wild type parasites with intact de novo pyrimidine synthesis pathways.

The cps1-1 and cps2-1 mutants were examined for virulence in balb/c mice. An ip administered inoculum of 100 parasites of either cps1-1 or cps2-1 had no measured virulence in balb/c mice, compared to the same dose of RH, cps1 or cps2 which were virulent. The avirulence of cps1-1 and cps2-1 correlates well with the pyrimidine dependence of parasite growth in vitro. The high concentrations of pyrimidines needed for growth of mutant cps1-1 and cps2-1 are simply not available in mammals such as mice. Other mammals including humans and other vertebrates are also not expected to have sufficiently high pyrimidine concentration to support growth of these mutants.

Higher dose virulence studies were also performed for the mutant cps1-1. Pyrimidine auxotroph cps1-1 was completely avirulent in Balb/c mice at doses equal to or greater than 10⁷ parasites delivered by ip inoculation.

The ability of the pyrimidine auxotroph mutants of the present invention to protect against infections was demonstrated. After 40 days ip inoculation of cps1-1 in balb/c mice, the same group of 4 mice were challenged with 200 parasites of the virulent RH strain (a 100% lethal dose) Mice originally inoculated with 10⁷ or 10⁵ cps1-1 parasites were completely protected from RH challenge. In contrast mice receiving only the lowest dose of 10³ cps1-1 parasites were completely unprotected from this RH parasite challenge. Thus, the pyrimidine auxotroph mutant given at appropriate dose is capable of protecting mice from lethal RH parasite challenge.

Safety of the pyrimidine auxotroph mutants of the present invention was also evaluated. A group of balb/c mice were inoculated with 10⁸ cps1-1 parasites and all survived at least 24 days post inoculation. A more rigorous test of safety was performed in immunocompromised mammals. Before the present invention, no T. gondii mutant had been isolated that would itself not kill gamma interferon homozygous knock-out mice (gko mice) (Jackson Labs, strain JR2286) (Radke, J R and White M W. Infection and Immunity 1999 67:5292-5297). Gamma interferon homozygous knock-out mice were inoculated with various doses of the pyrimidine auxotroph cps1-1 or a lethal low dose of RH parasites. Doses of cps1-1 (ip administered) at 10², 10⁴ and 10⁶ did not kill any of the 4 gamma interferon homozygous knock-out mice in each group, whereas all mice receiving RH parasites died within 8 days. Mutant cps2-1, was also avirulent in homozygous gamma interferon knock-out mice. Thus, the pyrimidine auxotroph mutants of the present invention are the first described T. gondii parasite isolates that are completely attenuated even in severely immunocompromised mice. The cps1-1 and cps2-1 mutants attach and invade as efficiently as wild type RH parasites in the absence or presence of pyrimidine in vitro in HFF cells. Thus the growth defect seen in immunocompromised mice is due to a block in intracellular replication only.

The pyrimidine growth dependence of mutant cps1-1 on the pyrimidine salvage pathway was further documented in thymidine interference experiments. Mutant cps1-1 plaques well in 250 μM uracil or deoxyuridine, but not in 1000 μM thymidine. Since thymidine at 1000 μM is known to inhibit approximately 90% of the parasite nucleoside phosphorylase activity specific for cleavage of deoxyuridine (Iltzsch, M. H. J. Euk. Micro. 1993 40:24-28), growth of cps1-1 was tested in combinations of 1000 μM thymidine and 250 μM uracil or 250 μM deoxyuridine. All pyrimidine salvage in T. gondii must pass through uracil and UPRT conversion of uracil to UMP. In contrast, thymidine has no effect on UPRT activity. Thus, if growth of cps1-1 were dependent on deoxyuridine supplementation then replication in this condition may be inhibited by co-supplementing with 1000 μM thymidine. It was found that thymidine did block deoxyuridine dependent growth of cps1-1 in these experiments by inhibiting nucleoside phosphorylase. However, thymidine did not affect UPRT or uracil dependent growth of cps1-1. Thus, when cps1-1 is grown in deoxyuridine the parasite strictly requires nucleoside phosphorylase activity to cleave deoxyuridine for growth. These data show cps1-1 and cps2-1 to have a marked defect in de novo pyrimidine synthesis and depleted UMP pools. Thus, cps1-1 and cps2-1 rely strictly on pyrimidine salvage enzymes for growth and further, the HFF host cell in vitro cannot supply sufficient pyrimidines for growth of cps1-1 or cps2-1. These data indicate that the pyrimidine auxotroph mutants cps1-1 and cps2-1 can be used in screening assays to identify compounds as inhibitors of various salvage enzymes when the replication of T. gondii is dependent on salvage pathways. Such potential inhibitors can only be identified using a pyrimidine auxotroph such as provided in the instant invention.

Survival, persistence, and reversion potential of pyrimidine auxotroph mutants cps1-1 and cps2-1 were also assessed. Ability of T. gondii mutants cps1-1 and cps2-1 to survive and persist intracellularly was determined in an in vitro survivability assay. From microscopic examination of cps1-1 and cps2-1, it is known that in the absence of pyrimidine addition the mutants attach and invade at normal efficiency and a single parasite can be observed in a small vacuole. With no pyrimidines added to growth medium the single parasite in the small vacuole remains as a non-replicated single parasite indefinitely. After 2 days of pyrimidine starvation, typically one bright blue (translucent) circular structure or 2 structures about 1 micron in diameter become apparent in many parasites and in most parasites by day 3 to day 4 of pyrimidine starvation. Thus, an assay was devised to measure whether the single non-replicating parasites inside the host cells were viable or non-viable. HFF flasks were inoculated with various parasite doses. At t=0 hours medium was changed, and pyrimidine starvation started. Then, at various time points (in days), cultures of pyrimidine starved cps1-1 and cps2-1 parasites were “rescued” by addition of 300 μM uracil. Incubation of rescued cultures was performed for 7 days in a plaque assay. All cultures were then examined for evidence of small micro-plaques by microscopic examination. Cultures were also stained for normal plaques and plaques were counted. The data from this assay indicates that parasites rapidly lose viability (loss of pyrimidine rescue). This loss roughly correlates with the appearance of the small bright blue circular structure in the intracellular non-replicating parasite that is starved of pyrimidines. Thus, simple culture of cps1-1 and cps2-1 in normal growth medium results in a pyrimidine starvation that efficiently kills intracellular cps1-1 and cps2-1 parasites. Thirty-two days of pyrimidine starvation was sufficient to kill at least 10⁶ PFU which was added to a single 25 cm² HFF flask. In these experiments it was also shown that addition of more than 2×10⁷ cps1-1 or cps2-1 parasites (MOI>10 parasites per HFF cell) to a single 25 cm² HFF flask resulted in all HFF cells becoming multiply infected with parasites each being a single parasite in an individual vacuole. Surprisingly even at these high MOI's of infection of HFF the host cell appeared perfectly normal, other than having 5 to 10 parasites within each cell on average. Thus, cps1-1 and cps2-1 also provide a useful strain of T. gondii to further analyze host-parasite interaction biology of obligate intracellular parasites. As demonstrated herein, these strains are particularly useful in further cell biological evaluation of the pyrimidine starvation phenotype or death phenotype.

To assess reversion, 106 to 10⁷ cps1-1 or cps2-1 parasites were periodically inoculated into HFF flasks supplemented with 5 μM uridine. This concentration of uridine is sufficient to rescue the parent strains (cps1 and cps2) of cps1-1 and cps2-1. However, it is insufficient to support any growth of cps1-1 or cps2-1. In multiple experiments involving a total of 5×10⁸ to 1×109 parasites of cps1-1 and cps2-1, no revertants were observed.

CPSII enzyme activity and thymidine kinase activity in parasite protein extracts derived from mutant or wild type parasites were also assessed. In these experiments, parasites were grown under appropriate conditions in multiple 25 cm² flasks or in 150 cm² flasks until lysis of the host monolayer. Extracellular parasites were purified through 3 micron nucleopore filters and parasites in parasite pellets were lysed in the presence of protease inhibitors to generate protein extracts for enzyme assays. The cpsII and TK enzyme activities in the various parasite extracts was determined in enzyme assays. The enzyme activity data indicates that the cps1 and cps2 mutants are partial knock-outs of cpsII activity compared to the activity measured in the wild type RH strain. Furthermore, as the bulk of data from pyrimidine rescue experiments indicated, no cpsII activity was detected in pyrimidine auxotroph mutants cps1-1 and cps2-1. Measurement of TK activity corresponded with previously determined sensitivity to ganciclovir. Parasites that were sensitive to ganciclovir had TK activity, whereas parasites that became resistant to ganciclovir lost TK activity. These measurements of cpsII enzyme activity confirm that the molecular defect of the cps mutants is primarily that of a loss of cpsII activity which results in blocking de novo synthesis of pyrimidines.

The sequences obtained from the cDNA and gDNA clones of T. gondii CPSII indicate that T. gondii has a CPSII organized like other apicomplexan CPSII enzymes. Accordingly, the same reasoning used to produce the T. gondii mutants is applicable to the generalized construction of pyrimidine auxotroph mutants in other apicomplexan parasites. Pyrimidine auxotroph mutants of other apicomplexan parasites are expected to provide protection against infection by apicomplexans in similar fashion to the T. gondii mutants exemplified herein. Thus, these apicomplesan pyrimidine auxotroph mutants can be administered to animals to immunize them against infection by apicomplexans.

To test the ability of the avirulent uracil auxotrophs of T. gondii to be act as a vaccine, the long-term protective immunity of the mutants was tested in vivo. BALB/c mice were challenged with a lethal dose of strain RH (200 tachyzoites) 40 days after inoculation with a single, intraperitoneal dose of the cps1-1 mutant strain of the present invention. The doses of the mutant strain were either 10³, 10⁴, 10⁵, 10⁶, or 10⁷ tachyzoites. When doses of the mutant exceeded 10,000 (10⁴) or more viable tachyzoites, long-term protective immunity was observed in the mice. In contrast, inoculation of mice with doses of less than 10,000 tachyzoites was much less effective at inducing a protective immune response in BALB/c mice. At a dose of 10³ mutant tachyzoites, there was zero percent survial at 10 days. At a dose of 10⁴ tachyzoites, survival was increased to 80 percent even out to 40 days. Survival was 100% at the three highest doses tested. When immune compromised mice were tested, although GKO mice still died after challenged with RH parasites, doses of 10⁶ or 10⁷ cps1-1 tachyzoites 30 days before challenge increased survial of the mice from 9 days (no cps1-1 treatment) to 11 days. These data demonstrated that an avirulent, easily produced, uracil auxotroph of T. gondii was a highly effective vaccine in mice and was able to confer protective immunity in immune competent animals.

The following nonlimiting examples are provided to further illustrate the present invention.

### EXAMPLES

### Example 1

### Parasite Material

Most of the work described herein used the RH strain of Toxoplasma gondii which is the most commonly used laboratory strain amongst Toxoplasma researchers (Pfefferkorn et al. Exp. Parasitol. 1976 39:365-376). Due to its long history of continuous passage in the laboratory, this strain is highly virulent in animals and grows rapidly in culture making it ideal for obtaining large amounts of material. However, it has lost the ability to go through the complete sexual cycle in cats. Parasites were grown in vitro in monolayers of cultured human foreskin fibroblasts (HFF) in accordance with well known procedures, for example, Fox et al. Mol. Biochem. Parasitol. 1999 98:93-103. Typically, using the RH strain, infected cultures were maintained by seeding uninfected monolayers; at about a 1:50 dilution every 48-72 hours. This yields about 10⁹ parasites from three T175 flasks of infected cultures. Parasites were harvested just as host lysis occurred filter purifying parasites through 3 micron nucleopore filters. Detailed methods for growth, harvesting, passage, purification of tachyzoite parasites, storage, and replication assays of T. gondii are routine and well known, for example, Roos et al. Meth. Microb. Path. 1994 45:23-65, and Fox et al. Mol. Biochem. Parasitol. 1999 98:93-103. All media reagents were purchased from Gibco-BRL (Rockville, Md.), Bio Whittaker (Walkersville, Md.) and Sigma (St. Louis, Mo.).

### Example 2

### Chemicals and Enzyme Assays

Most chemical or biochemical reagents were purchased from Sigma (St. Louis, Mo.). Ganciclovir was obtained from Roche Labs, Nutley, N.J. The linked assay system for cpsII activity was performed in accordance with known methods, for example, Asai et al. Mol. Biochem. Parasitol. 1983 7:89-100 and Hill et al. Mol. Biochem. Parasitol. 1981 2:123-134. For cpsII assays parasites were lysed in M-PER extraction buffer (Pierce Inc., Rockford, Ill.) or by osmotic shock in 4 volumes (w/v) of 10 mM potassium phosphate (pH 7), 0.05 mM dithiothreitol and protease inhibitors antipain, leupeptin, chymostatin, and pepstatin A, each at 0.1 mM. After 1 to 2 minutes of lysis, glycerol 7.5% (w/v) was added back to the extracts. The lysed parasite extracts were centrifuged at 20,000× g for 15 minutes and the supernatants used in cpsII enzyme assay. CpsII reaction assays contained 50 mM HEPES (pH 7.2), 10% (w/v) glycerol, 20 mM MgCl₂, 20 mM ATP, 3 mM L-glutamine, 0.5 mM L-ornithine, 10 mM KCl, 0.05 mM dithiothreitol, 1 unit ornithine carbamyl transferase, 10 mM bi[¹⁴C]carbonate(1 μCi/μM) and extract in a final volume of 0.1 ml. Sodium bi[¹⁴C]carbonate was 30-60 mCi/mmol and obtained from ICN. Reactions were run for 30 minutes at 37° C. and then terminated by addition of 10 μl of 5 M formic acid. A small piece of solid CO₂ was dropped into the stopped reaction and excess CO₂ was removed by standing the open solution in a fume hood. Reaction volumes were removed, dried and redissolved in 0.1 ml water prior to addition of liquiscint scintillant and counting of [¹⁴C] in a Beckman scintillation counter.

Thymidine kinase assays were performed in accordance with known methods, for example, Maga et al. (Biochemical Journal 1994 302:279-282). Briefly, parasite extracts were lysed by sonication (1 minute, kontes microtip) or in M-PER extraction buffer plus a cocktail of protease inhibitors (antipain, leupeptin, chymostatin, and pepstatin A; 10 μg each) and 1 mM phenylmethylsulfonyl fluoride. TK assays were run in a 50 μl volume at 37° C. for 30 minutes in a mixture containing 30 mM potassium HEPES (pH 7.5), 6 mM ATP, 6 mM MgCl₂, 0.5 mM dithiothreitol and 3.3 μM [³H]Thymidine (20-40 Ci/mmol from ICN). The reaction was terminated by transferring 25 μl of the incubation mixture to DE81 ion exchange paper (Whatman, Clifton, N.J.). The spotted paper was washed in 1 mM ammonium formate (pH 3.6) to remove unconverted nucleoside, distilled water, and then a final ethanol wash prior to drying of the paper and scintillation counting in liquiscint. Protein determination for parasite protein extracts was determined using Bio-Rad protein assay reagents and bovine serum albumin in accordance with standard procedures (Bio-Rad, Hercules, Calif.).

### Example 3

### Molecular Methods

Molecular methods including DNA isolation, restriction, Southern blotting, hybridization, and PCR reactions used herein are all well known, for example, Bzik et al. Proc. Natl. Acad. Sci. USA 1987 84:8360-8364 and Fox et al. Mol. Biochem. Parasitol. 1999 98:93-103. Transfection of T. gondii was also performed in accordance with routine procedures (Roos et al. Meth. Microb. Path. 1994 45:23-65 and Fox et al. Mol. Biochem. Parasitol. 1999 98:93-103). The gene libraries were developed from HindII or PstI digested genomic DNA cloned into bluescript KSII digested with the same enzyme and treated with alkaline phosphatase prior to ligation with T. gondii DNA fragments. Libraries were manipulated in accordance with known methods (Bzik et al. Proc. Natl. Acad. Sci. USA 1987 84:8360-8364). Total mRNA was isolated from T. gondii using TRIZOL-LS reagent (Gibco-BRL, Rockville, Md.) and mRNA was converted to cDNA using a cDNA kit from Pharmacia (Piscataway, N.J.) with polydT or random hexamers primers. DNA sequencing was done using classical dideoxy chain termination or automated sequencing using fluorescent dyes (ABI sequencer, Foster City, Calif.). DNA sequences were analyzed using the MacVector suite of programs (Oxford Molecular, Beaverton, Oreg.) and resources at NCBI, such as blast search. The DHFRm2m3-TS allele was obtained from the NIH AIDS Reference and Reagent Center (Rockville, Md.). The TK75 allele which was described by Black et al. Proc. Natl Acad. Sci. USA 1996 93:3525-3529 was obtained from Darwin (Seattle, Wash.). Bluescript plasmid was from Stratagene (La Jolla, Calif.). Restriction enzymes, nucleic acid modifying enzymes and transfer membranes were from Boehringer Mannheim, Indianapolis, Ind.

### Example 4

### Experimental Infection and Animal Studies

Balb/c inbred mice and balb/c mice bearing a homozygous knock-out of interferon gamma (gko) were obtained from Jackson Labs (Bar Harbor, Me.). Tachyzoite parasites were aseptically handled and purified from freshly lysed monolayers of infected HFF cells through a sterilized 3 micron polycarbonate membrane (Nucleopore, Cambridge, Mass.). Parasite concentration was scored microscopically in a hemocytometer. Purified parasites were pelleted at 1500 g for 10 minutes and washed in sterile EMEM media with no supplements and without disturbing the parasite pellet. The centrifuge tube was centrifuged once more for 2 minutes and the supernatant removed and replaced with EMEM media containing no supplements in a volume of EMEM to give a 10 times higher concentration (per/ml) of parasites than the highest dose. This was done so inoculation of 0.1 ml of this solution would equal the highest parasite dose. Parasites were gently resuspended in sterile EMEM (no additions). Mice, in groups of four, were inoculated with appropriate doses of tachyzoite parasites in EMEM. Following inoculation of mice the residual volume of unused tachyzoite parasites was returned to the sterile hood and dilutions were made to represent 200 and 400 parasite plaques on 25 cm² HFF flasks assuming 100% recovery of parasites after centrifugation/resuspension and 100% percent viability. Then, following a 7 day plaque assay, actual plaques were counted, post-inoculation of mice, and the percent viable PFU ratio to parasite counts in the hemocytometer were determined microscopically in every experimental infection. Uniformly, all of the mutants described herein as well as RH parasites always fell in the range of 0.4 to 0.6 viable PFU per parasite counted using these conditions. Following inoculation of mice, mice were observed daily for signs of infection (or distress) or death.

What is claimed is:

1. An isolated nucleic acid sequence encoding carbamoyl phosphate synthase of T. gondii.

2. The nucleic acid sequence of claim 1 comprising SEQ ID NO:1 or SEQ ID NO:2.

3. A pyrimidine auxotroph mutant of an apicomplexan wherein one of six enzymes of the de novo pyrimidine biosynthesis pathway of apicomplexans is mutated.

4. The pyrimidine auxotroph mutant of claim 3 wherein the apicomplexan is T. gondii.

5. A method for protecting an animal against infection by an apicomplexan comprising administering to an animal a pyrimidine auxotroph mutant of claim 3.

6. The method of claim 5 wherein the apicomplexan is T. gondii.

7. A method for screening for inhibitors of pyrimidine salvage enzymes in apicomplexans comprising: contacting a pyrimidine auxotroph mutant of claim 3 with a compound suspected of being an inhibitor of a pyrimidine salvage enzyme; and determining growth of the pyrimidine auxotroph mutant in the presence of the compound, wherein inhibition of growth of the mutant is indicative of the compound being an inhibitor of a pyrimidine salvage enzyme.

8. A vaccine for protection against infection by an apicomplexan comprising the pyrimidine auxotroph mutant of claim 3 and a pharmaceutically acceptable carrier or diluent.
